(19) United States
(12) Patent Application Publication
(10) Pub. No.: US 2009/0264533 A1
(43) Pub. Date: Oct. 22, 2009

(54) Methods and Compositions for Modulating RHO-Mediated Gene Transcription

(76) Inventors: Richard Neubig, Ann Arbor, MI (US); Chris Evelyn, Wheatley Heights, NY (US)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 11/910,383

(22) PCT Filed: Mar. 21, 2006
(86) PCT No.: PCT/US2006/010089

§ 371 Date: Apr. 1, 2008

Related U.S. Application Data
(60) Provisional application No. 60/666,374, filed on Mar. 30, 2005.

(51) Int. Cl. A61K 31/18; C07C 311/00; C07C 237/00; A61K 31/16; C12N 5/02

(52) U.S. Cl. 514/603; 564/86; 564/155; 514/616; 435/375

(57) ABSTRACT

The present invention is directed to methods and compositions methods and compositions for determining and inhibiting gene transcription in mammalian cells. The invention relates to novel inhibitors of rho-mediated gene transcription and to compounds that may be used as therapeutic agents.

Some of the work presented herein was supported by grant funding from the National Institutes of Health Grant No. GM39561.

### FIELD OF THE INVENTION

The present invention is in the field of gene transcription in mammalian cells. More particularly, the present invention is directed to methods and compositions for identifying modulators of rho-mediated gene transcription in mammalian cells.

### BACKGROUND

Cancer metastasis is a significant medical problem in the United States, where it is estimated that >500,000 cancer-related deaths in 2003 were as a result of metastatic tumors rather than effects of the primary tumor (approximately 90% of cancer deaths). Cancer metastasis is a result of malfunction in several tightly regulated cellular processes that control cell movement from a primary site to a secondary site. These cellular processes include cell survival, adhesion, migration, proteolysis as it pertains to extracellular matrix remodeling, immune escape, angiogenesis and lymphangiogenesis, and target ‘homing.’ Most existing treatments of cancers have focused on killing tumor cells. The problem with such chemotherapeutic intervention is that it leads to substantial toxicity. Since spread, or metastasis, of cancers is the primary way in which cancer kills people, it would be desirable to find agents that inhibit or prevent signals that trigger metastasis.

It has been shown that rho proteins are overexpressed in various tumors, including colon, breast, lung, testicular germ cell, and head and neck squamous-cell carcinoma (Sawyer, Expert Opin. Investig. Drugs., 13: 1-9, 2004). The rho family of small GTP binding proteins plays important roles in many normal biological processes and in cancer (Schmidt and Hall, Genes Dev., 16:1587-1609, 2002; Burridge and Wennerberg, Cell, 116:167-179, 2004). This family includes three main groups (rho, rac, and cdc42). Rho itself is activated by numerous external stimuli including growth factor receptors, immune receptors, cell adhesion, and G protein coupled receptors (GPCRs) (Schmidt and Hall, Genes Dev., 16:1587-1609, 2002, Sah et al., Annu. Rev. Pharmacol. Toxicol., 40:459-489, 2000).

Several reports have suggested an important role for rhoA and/or rhoC in metastasis (Clark et al., Nature 406:532-535, 2000; Ikoma et al., Clin Cancer Res 10:1192-1200, 2004; Shikada et al., Clin Cancer Res 9:5282-5286, 2003; Wu et al., Breast Cancer Res Treat 84:3-12, 2004.) Both rhoA and rac1 can regulate the function of the extracellular matrix (ECM) proteins: ezrin, moesin, and radixin, by the phosphorylation of ezrin via the rhoA pathway and the phosphorylation of the ezrin antagonist, neurofibromatosis 2, by the rac1 pathway (Shaw et al., Dev Cell 1:63-72, 2001; Matsui et al., J Cell Biol 140:647-657, 1998). These ECM proteins are known to promote cell movement by utilizing the ECM receptor, CD44, to link the actin cytoskeleton with the plasma membrane. In addition, rhoA and rac1 can regulate ECM remodeling by controlling the levels of matrix metalloproteinases (MMPs) or their antagonists, tissue inhibitors of metalloproteinases (TIMPs, Bartolome et al., Cancer Res 64:2534-2543, 2004). RhoA has also been reported to be required for monocyte tail retraction during transendothelial migration suggesting a role in extravasation (Worthylake et al., J Cell Biol 154:147-160, 2001) which is a key process in metastasis.

In addition to cytoskeletal effects, rhoA induces gene transcription via the serum response factor, SRF and SRF has been associated with cellular transformation and epithelial-mesenchymal transformation (Iwahara et al., Oncogene 22:5946-5957, 2003; Psichari et al., J Biol Chem 277:29490-29495, 2002). Rho activates SRF by a mechanism that involves release of the transcriptional coactivator, megakaryoblastic leukemia protein—MKL (Cen et al., Mol Cell Biol 23:6597-6608, 2003; Miralles et al., Cell 113:329-342, 2003; Selvaraj and Prywes, J Biol Chem 278:41977-41987, 2003). MKL (like the rhoGEF LARG) was first identified as a site of gene translocation in leukemia (megakaryoblastic leukemia) (Mercher et al., Genes Chromosomes Cancer 33:22-28, 2002). A recent paper showed that the protein product of the translocated MKL gene is hyperactive compared to the wild-type protein. MKL has also been called modified in acute leukemia—MAL or BSAC (Miralles et al., Cell 113:329-342, 2003; Sasazuki et al., J Biol Chem 277:28853-28860, 2002). Interestingly MKL/BSAC was recently identified in an antiapoptosis screen for genes that abrogate tumor necrosis factor-induced cell death (Sasazuki et al., J Biol Chem 277:28853-28860, 2002). As a consequence of rho signaling, MKL is translocated to the nucleus and binds SRF leading to the expression, among other things, of c-fos which along with c-jun forms the transcription factor AP-1 promoting the activity of various MMPs and other cell motility genes (Benbow and Brinckerhoff, Matrix Biol 15:519-526, 1997). These genes can potentially lead to cancer cell invasion and metastasis. Overall, these findings draw a link between rho controlled biological processes and the processes involved in cancer metastasis. Similarly both LARG and MKL appear to be important players in these processes.

There is, however, substantial confusion in the literature on the relative contributions of rho and rac proteins in the metastatic phenotype (Sahai and Marshall, Nat Rev Cancer 2:133-142, 2002; Whitehead et al., Oncogene 20:1547-1555, 2001). A recent paper by Sahai and Marshall (Nat Cell Biol 5:711-719, 2003) sheds light on this conundrum. Those authors documented that different tumor cell lines exhibit different mechanisms of motility and invasion. In particular 375 m2 melanoma and LS174T colon carcinoma cell lines showed a striking “rounded” and “blebbed” morphology during invasion into Matrigel matrices. This invasion was entirely rho-dependent in that the invasion was blocked by C3 exotoxin, the N17rho dominant negative protein, and a ROCK kinase inhibitor. In contrast, two other cell lines were blocked instead by a rac dominant negative but not rho or ROCK inhibitors. These latter two cells (BE colon carcinoma and SW962 squamous cell carcinoma) had a distinct elongated morphology while a third line showed a mixed morphology and was blocked partially by both rho and rac inhibitors. Clearly there is important heterogeneity in mechanisms of tumor cell behavior that may contribute to metastasis. It is widely recognized that cell growth and apoptosis mechanisms vary greatly among tumors and that any targeted therapeutic approaches will need to take this into account.

Thus, development of targeted therapies for cancer will depend on an understanding of critical molecular steps in specific tumors. Signaling by growth factors, G protein coupled receptors, and small GTP binding proteins are well-known to play important roles in the biology of cancer. In some tumors, rho family small GTP binding proteins are strongly implicated in cell transformation and metastasis. In particular, RhoA and RhoC are upregulated in melanoma and in breast, lung, prostate, and pancreatic cancer. Expression of RhoA and/or RhoC has been correlated with tumor aggressiveness, invasiveness, and metastasis and Rho plays a functional role in animal models and some human cancers. In addition, thrombin, lysophosphatidic acid (LPA), and other agonists at G protein coupled receptors are associated with changes in cell motility, invasion, and metastatic behavior.

A key principle in targeted therapies is that only those tumors utilizing a particular signaling pathway will be susceptible to such therapy. Thus, defining the molecular components of these signaling pathways and their roles in various tumors will be critical to the appropriate use of rho-targeted therapies. The design and identification of novel chemical inhibitors of these pathways should contribute to the armamentarium of targeted cancer therapies. The present application defines pathways involved in rho-dependent cellular behaviors that are related to metastasis and provides methods and compositions for inhibiting such pathways.

### SUMMARY OF THE INVENTION

The present invention is directed to compositions and methods for the treatment of disorders that involve rho-signaling. More specifically, the present invention is directed to compositions for inhibiting rho-mediated gene transcription, where the compositions comprise an isolated compound that has a general structural formula (I):

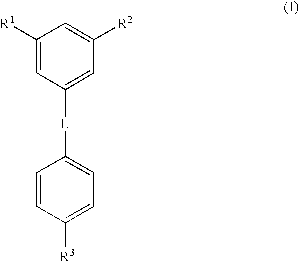

wherein R¹ and R², independently are selected from the group consisting of hydrogen, halo, C₁₋₃allyl, C₁₋₃alkoxy, CF₃, OCF₃, C₁₋₂alkylOCH₂, NO₂, and CN, with the proviso that at least one of R¹ and R² is different from hydrogen;

R³ is selected from the group consisting of halo, C₁₋₃alkyl, C₁₋₃alkoxy, CF₃, OCF₃, C₁₋₂alkylOCH₂, NO₂, and CN: and

L is a linking group about four to about eight atoms or functional groups in length, wherein said compound is present in an amount effective to inhibit rho-mediated gene transcription.

In particular embodiments, the isolated compound of general formula I comprises two terminal hydrophobic phenyl groups, and a hydrophilic linking group L. In certain preferred embodiments, L is selected from the group consisting of C(═O), C(═S), NR^{a}, C═N(R^{a}), SO₂, SO, O, S, a phenyl ring and C(R^{b})₂ group, wherein R^{a} independently, is hydrogen, hydroxy, or methyl and wherein R^{b}, independently, is hydrogen or methyl.

In specific embodiments, the isolated compound has the formula A or formula B:

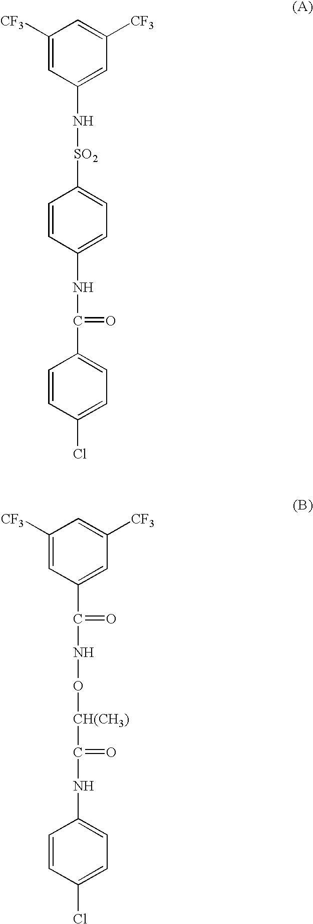

Also encompassed herein are pharmaceutical compositions that comprise an isolated compound described above and a pharmaceutically acceptable carrier, excipient or diluent.

Another aspect of the invention is directed to a method of inhibiting MKL-dependent gene transcription, SRF-dependent gene transcription, rho-stimulated gene transcription, LPA-stimulated DNA synthesis, or spontaneous matrix invasion of a cancer cell comprising contacting said cancer cell with such an isolated compound. More specifically, methods of inhibiting cancer cell growth comprising contacting a cancer cell with such an isolated compound are contemplated. In some embodiments, cancer cell may be located in vitro and the inhibition of cancer cell growth is in an in vitro assay. In other embodiments, the cancer cell is located in vivo in an animal. In such in vivo embodiments, the cancer cell is in a tumor and said inhibition of cancer cell growth comprises a decrease in tumor size. The cancer cell preferably is a metastatic cancer cell and said inhibition comprises a reduction in metastatic spread of said cancer cell. By metastatic cancer cell, the present invention is referring to a cancer cell that can metastasize. The compositions of the present invention are useful in that they inhibit or prevent a cancer cell from going into metastases. In specific embodiments, the cancer cell is a cell in which rho proteins are overexpressed, including but not limited to colon cancer, breast cancer, lung cancer, testicular germ cell cancer, and head and neck squamous-cell carcinoma. In specific embodiments, the tumor is resected and the composition is contacted with said tumor prior to resection or during resection or said composition is contacted with said animal at the cavity of said tumor resection. In some embodiments, the cancer cell is in a tumor and said inhibition of cancer cell growth comprises a decrease in tumor size. In specific preferred embodiments, the invention is directed to preventing or inhibiting metastasis of a potentially metastatic cancer cell. Such a potentially metastatic cancer cell may be part of a tumor. Where the cancer cell or the potentially metastatic cancer cell is in a tumor, the method of the invention may be used when the tumor is resected wherein the composition is contacted with said tumor prior to resection or during resection or said composition is contacted with said animal at the cavity of said tumor resection.

Other embodiments contemplate treating an inflammatory response in an animal comprising contacting or administering to the animal with an isolated compound of the invention. The inflammatory response to be treated may be any inflammatory response that causes an inflammatory disease or disorder. Exemplary such diseases or disorders include, for example asthma, atopic dermatitis, allergic rhinitis, systemic anaphylaxis or hypersensitivity responses, drug allergies (e.g., to penicillin, cephalosporins), insect sting allergies and dermatoses such as dermatitis, eczema, atopic dermatitis, allergic contact dermatitis, urticaria, rheumatoid arthritis, osteoarthritis, inflammatory bowel disease e.g., such as ulcerative colitis, Crohn's disease, ileitis, Celiac disease, nontropical Sprue, enteritis, enteropathy associated with seronegative arthropathies, microscopic or collagenous colitis, eosinophilic gastroenteritis, or pouchitis resulting after proctocolectomy, and ileoanal anastomosis, disorders of the skin [e.g., psoriasis, erythema, pruritis, and acne], multiple sclerosis, systemic lupus erythematosus, myasthenia gravis, juvenile onset diabetes, glomerulonephritis and other nephritides, autoimmune thyroiditis, Behcet's disease and graft rejection (including allograft rejection or graft-versus-host disease), stroke, cardiac ischemia, mastitis (mammary gland), vaginitis, cholecystitis, cholangitis or pericholangitis (bile duct and surrounding tissue of the liver), chronic bronchitis, chronic sinusitis, chronic inflammatory diseases of the lung which result in interstitial fibrosis, such as interstitial lung diseases (ILD) (e.g., idiopathic pulmonary fibrosis, or ILD associated with rheumatoid arthritis, or other autoimmune conditions), hypersensitivity pneumonitis, collagen diseases, sarcoidosis, vasculitis (e.g., necrotizing, cutaneous, and hypersensitivity vasculitis), spondyloarthropathies, scleroderma, atherosclerosis, restenosis and myositis (including polymyositis, dermatomyositis), pancreatitis and insulin-dependent diabetes mellitus.

Also contemplated is the use of a compound or composition of the invention for the treatment of a disease that is mediated through rho-dependent gene transcription. Another aspect contemplates the use of a composition of the invention in the manufacture of a medicament for the treatment of a disease that is mediated through rho-dependent gene transcription. Alternative embodiments contemplate the use of a composition of the invention in the manufacture of a medicament for the treatment of metastatic cancer cell wherein said composition produces a reduction in metastatic spread of said cancer cell.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWING(S)

The following drawings form part of the present specification and are included to further illustrate aspects of the present invention. The invention may be better understood by reference to the drawings in combination with the detailed description of the specific embodiments presented herein.

FIG. 1. Gα13/LARG/SRE Transcriptional signaling pathway involved in the luciferase assay.

FIG. 2 Optimization of HTS Assay. HEK293T cells in 10 cm dishes were co-transfected with 2×-SRE luciferase reporter plasmid (3000 ng) alone or with Gα13QL (5 ng constitutive active mutant) and LARG (150 ng) expression plasmids for 5 hours. Then, the cells were plated into 96-well, and serum-starved for approx. 18 hours (0.5% Fetal Bovine Serum, DMEM medium). Cells were lysed and assayed for luciferase activity via Steady-Glo reagent (Promega) and a luminometer. The Z-score was calculated from the assay to be approx. 0.7, which indicates that the assay is an “excellent assay” for HTS.

FIG. 3 MKL-DN inhibits SRE Luciferase expression activated by rho pathway signals. HEK293T cells were transfected with the SRE.L Luciferase reporter, CMV-Renilla control reporter and the indicated activators with or without MKL1-DN. MIL-DN suppresses rho-dependent transcription (F/R ratio) with minimal effects on CMV-Renilla expression.

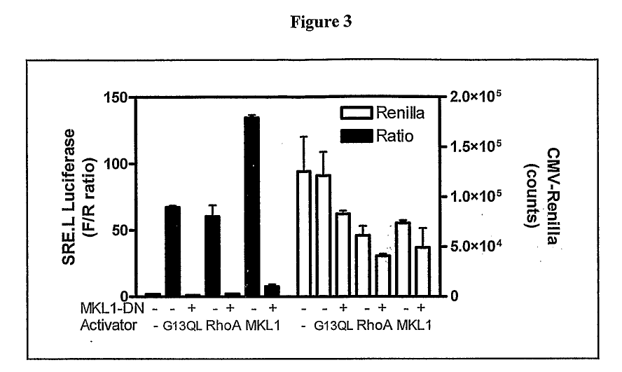

FIG. 4 Chemical Structures of CCG-977 (left) and CCG-1423 (right). There is a striking similarity between the structures of the two compounds that showed specific inhibition of rho-stimulated gene transcription. These compounds share the 1,3 bis-trifluoromethyl benzene and the chlorophenyl moieties.

FIG. 5 Inhibition of G13/LARG-stimulated rho transcriptional signaling by Latrunculin B, the rho kinase inhibitor Y-27632 and CCG-1423 one of the two compounds identified in our high throughput screen for rho pathway inhibitors.

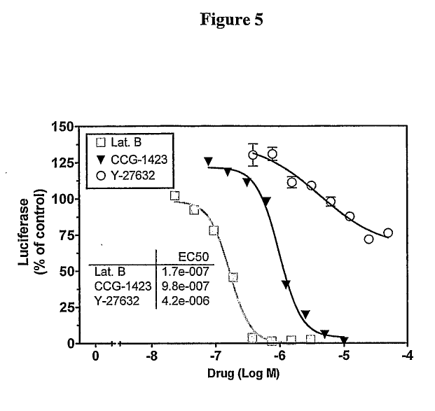

FIG. 6 CCG-1423 inhibits both RhoA and RhoC transcriptional signals. Cells were transfected with the dual reporters SRE-Luciferase and CMV-Renilla then 6 hours later, 6 micromolar CCG-1423 was added and reporter activity measured 16 hours later.

FIG. 7 Signaling mechanisms that activate rho.

FIG. 8 Inhibition of reporter gene transcription in PC-3 cells. CCG 1423 (10 μM) acts at the transcriptional level inhibiting SRE Luciferase stimulated by both upstream (Gα13, LARG, and RhoA-GV) and downstream signals (MKL1). It does not inhibit CMV-driven Renilla expression and has no effect on SRF-VP16 and GAL4-VP16 Luciferase.

FIG. 9 Serum and LPA stimulate DNA synthesis in PC-3 cells. PC-3 cells serum starved for 24 hours were treated for an additional 24 hours in the presence of serum (10% FBS) or the indicated concentrations of LPA. BrdU was added to the medium for the last 2 hours and incorporated BrdU detected in 96-well plates by immunostaining.

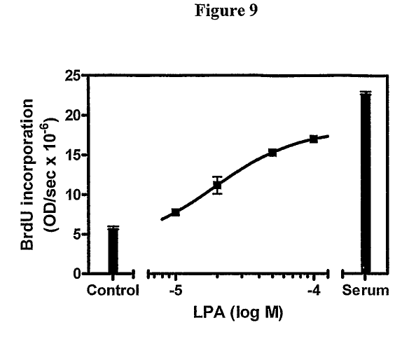

FIG. 10 CCG-1423 inhibits LPA- but not FBS-stimulated DNA synthesis in PC-3 prostate cancer cells. In addition to blocking rho-dependent transcriptional effects, CCG 1423 potently inhibits LPA-stimulated DNA synthesis (IC₅₀ 900 nM).

FIG. 11 CCG-1423 inhibits the spontaneous matrix invasion by PC-3 cells. Top) One prostate and three ovarian cancer lines (50,000 cells per well) were lifted with trypsin, suspended in serum free medium and plated on 24-well Matrigel inserts with (solid) or without (hatched) 30 μM LPA in the lower chamber. After 24 hours, cells above the filter were wiped off and the remaining cells were fixed, stained, and counted to determine the number of cells that had invaded the Matrigel. Bottom) Matrigel invasion by PC-3 cells (-LPA) and SKOV-3 cells (+30 μM LPA) was tested in the presence of the indicated concentrations of CCG-1423. The curve is a non-linear least squares fit with an IC₅₀ of 830 nM.

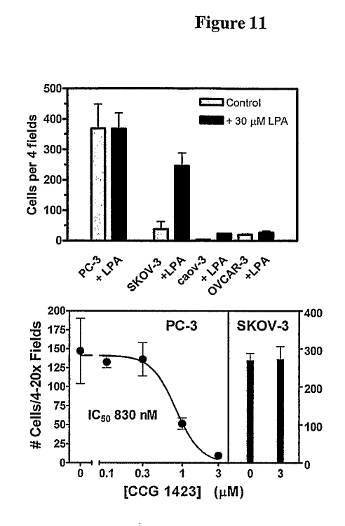

FIG. 12 CCG-1423 selectively inhibits PC-3 cell growth. PC-3 or SKOV-3 cells (2000 per well) were plated in medium containing 2% FBS. After growth overnight, the indicated concentrations of CCG-1423 were added from a DMSO stock (<1% final) and cells allowed to continue growing for an additional 7 days. Cell growth was measured by WST1 absorbance (similar to MTT). The IC₅₀ values for PC-3 and SKOV-3 cells were 600 nM and 3 μM. The dotted line represents a no-cell blank.

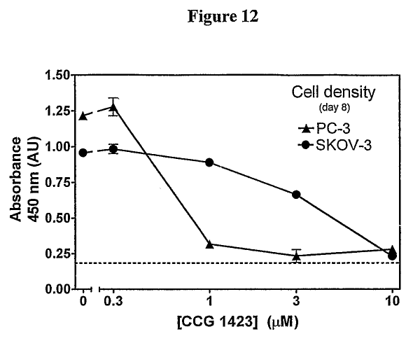

FIG. 13. Inhibition by CCG-1423 of three different rho pathway activators (G13, rhoA-GV, and MKL1) with no effect on control reporter plasmid CMV-Renilla.

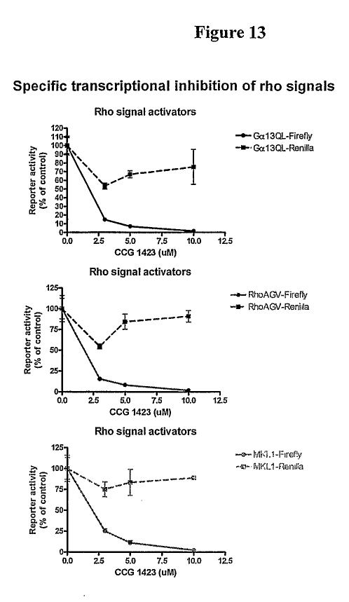

FIG. 14. Since CCG-1423 was able to inhibit SRF-VP16 stimulated luciferase expression we tested its ability to inhibit ras-stimulated transcriptional signals as well as rho. The Bottom panel shows inhibition of ras signals by CCG-1423 but not by Latrunculin B. This confirms the novel mechanism of CCG-1423. The rho reporter SRE.L can also be activated by ras (probably via rho activation). That signal is blocked by Lat B indicating that the SRE.L is primarily reading rho signals while c-fos detects both ras and rho.

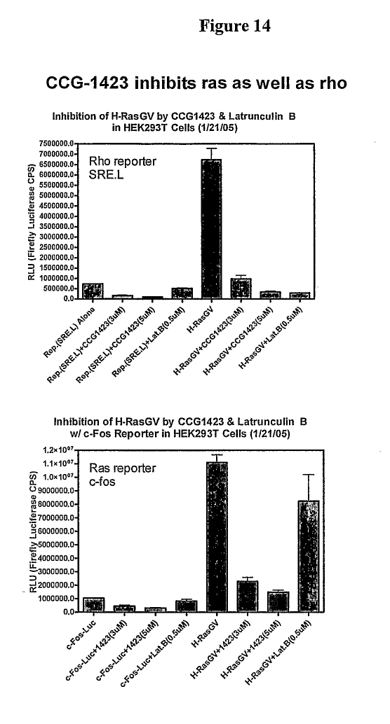

### DETAILED DESCRIPTION OF THE PREFERRED EMBODIMENTS OF THE INVENTION

Rho signaling is well-established to contribute to cancer cell proliferation, migration, gene expression, and metastasis. In the present invention, by obtaining a better understanding of the mechanisms and role of rho signaling in cancer cell function, the inventors have developed screens and methods of identifying agents that will act at key points in the pathway of rho signaling in the cancer phenotype as well as other rho-mediated phenotypes. Through this better understanding of the mechanisms of action of rho, the inventors have identified small molecule chemical inhibitors that disrupt the rho signaling pathways. In order to achieve these results, the inventors focused on the components of rho signaling involved in both upstream regulation (receptor and rhoGEF) and downstream regulation (especially rho-stimulated gene transcription) and designed a high-throughput chemical inhibitor screen of the rho pathway. One of the novel compounds that was identified using the methods described herein works at the level of gene transcription. This inhibitor, identified herein below as CCG-1423, at nanomolar concentrations selectively inhibits rho-stimulated gene transcription and also inhibits metastasis-related cancer cell behaviors such as DNA synthesis, proliferation, and matrix invasion. Methods and compositions for exploiting the new inhibitors identified herein, as well as for identifying additional related compounds that will be useful in modulating rho-mediated functions are contemplated as part of the present invention.

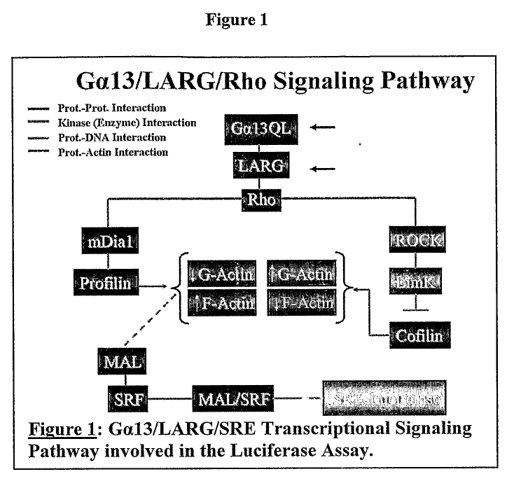

In the high throughput screening methods that are described in further detail herein below as part of the invention, the inventors were interested in compounds that could inhibit either upstream (receptor) or downstream (rho or gene transcription) signal. Therefore, the inventors used rho-dependent transcriptional reporter SRE.L Luciferase (Miralles et al., Cell 113:329-342, 2003; Mao et al., Proc Natl Acad Sci USA 95:12973-12976, 1998) in a cell-based high-throughput screening assay to identify small molecule inhibitors of the Gα13/LARG/rho/MKL transcriptional signaling pathway (FIG. 1). As noted in the Examples, with just a small screen (2000 compounds), it was possible to identify an 800 nM inhibitor of G13/LARG (and rho-) stimulated gene expression. This compound (CCG-1423) has also been found to selectively inhibit PC-3 cell proliferation and matrix invasion. The methods and compositions are described in further detail below.

### A. Involvement of Rho in Cell Signaling.

Throughout the present invention, there is discussion of the involvement of rho-dependent transcription in metastasis. The present discussion is provided by way of introduction as to the role of this rho in metastases and to provide definitions of certain terms used herein.

For purposes of the present invention, the term “rho” or “rho proteins” will represent the narrowly defined rho subfamily that includes rhoA, rhoB, rhoC, etc. and is described in (Sahai and Marshall, Nat Rev Cancer 2:133-142, 2002). These terms do not refer to the larger rho family (i.e. do not refer to rac and cdc42). The term “Rho family” will be used to designate the larger group including the three rho subfamilies (rho, rac, and cdc42).

The mechanism of signaling by heterotrimeric G protein-coupled receptors that activate rho has been obscure until recently (Sah et al., Annu Rev Pharmacol Toxicol 40:459-489, 2000). The discovery of a family of unique rho guanine nucleotide exchange factors (rhoGEFs), p115rhoGEF (Hart et al., J Biol Chem 271:25452-25458, 1996), PDZrhoGEF (Fukuhara et al., J Biol Chem 274:5868-5879, 1999), and LARG (Leukemia-associated rhoGEF, Kourlas et al., Proc Natl Acad Sci USA 97:2145-2150, 2000 suggested a simple mechanism. They contain a regulator of G protein signaling (RGS) domain that binds activated Gα12 (Suzuki et al., Proc Natl Acad Sci USA 100:733-738, 2003) and Gα13 (Hart et al., Science 280:2112-2114, 1998) causing rhoGEF activation. Thus, the RGS-rhoGEFs appear to serve as effectors of activated Gα12/13 and as molecular bridges between the heterotrimeric G protein alpha subunits and rho. This represents a novel action of an RGS-domain containing protein since they typically inhibit GPCR responses (Neubig and Siderovski, Nat Rev Drug Discov 1:187-197, 2002). A role for RGS-rhoGEF proteins in cellular rho signaling by GPCRs, such as those for thrombin and lysophosphatidic acid (LPA), has been suggested by studies with dominant negative constructs (Mao et al., Proc Natl Acad Sci USA 95:12973-12976, 1998; Majumdar et al., J Biol Chem 274:26815-26821, 1999) and inhibition of signaling by expression of the RGS-domains which act as Gα12/13 inhibitors (Fukuhara et al., FEBS Lett 485:183-188, 2000).

Despite this recent flurry of activity, there has not been direct proof that these RGS-RhoGEF proteins mediate GPCR signals and no information was available about which rhoGEF(s) are downstream of which receptors until the work of the inventors (Wang et al., J Biol. Chem., 279(28):28831-28834, 2004). Experimentally, rho activation can be detected directly by measurements of GTP-bound active rho precipitated from cell lysates with effector fusion proteins such as GST-rhotekin (Reid et al., J Biol Chem 271:13556-13560, 1996) or indirectly by any number of functional readouts. The 1321N1 astrocytoma cells is a well-studied model of thrombin-induced rho activation (Majumdar et al., J Biol Chem 273:10099-10106, 1998). Thrombin induces both cell rounding and enhanced cell proliferation in these astrocytoma cells by mechanisms that are independent of known second messengers but are blocked by rho inhibitors.

In Wang et al., (J Biol. Chem., 279(28):28831-28834, 2004) the inventors for the first time used HEK293T cells and an aggressive, metastatic, human prostate cancer cell line, PC-3, to test the role of the three RGS-rhoGEFs (LARG, p115-, and PDZrhoGEF) in receptor signaling. HEK293 and PC-3 cells express all three of these proteins with RNA for PDZrhoGEF and LARG being more abundant than that for p115. PC-3 cells are known to overexpress the thrombin receptor (PAR1) and have an increased propensity to metastasize to bone compared to lines that have lower PAR1 expression (Cooper et al., Cancer 97:739-747, 2003). To demonstrate a role for rhoGEFs in GPCR signaling and to define the specificity of their actions, 21 nt short interfering RNAs (siRNAs) targeting each of these RGS-rhoGEFs were prepared. It was shown that LARG mediates thrombin responses while the LPA response is mediated by PDZrhoGEF. This was the first direct demonstration of a role for an RGS-rhoGEF in G protein coupled receptor signaling. Furthermore, it pinpointed the RGS-rhoGEFs that were most important (LARG and PDZrhoGEF). The present application further for the first time provides methods and compositions that use rhoGEFs in methods for screening for modulators of rho-stimulated activities.

In the present application, the role of RGS-rhoGEFs in rho signaling by different receptors is further defined. The inventors developed synthetic RNAi molecules against the three members of this protein family. Using this approach, it is shown herein that in PC-3 cells, the thrombin receptor (PAR1) utilized LARG while the LPA receptor utilized PDZ-rhoGEF for inducing cell rounding (Wang et al., J Biol. Chem., 279(28):28831-28834, 2004). In addition, direct measurements of thrombin-induced rho activation in HEK293T cells (using GST-rhotekin pulldown) also showed a dependence on LARG. In the course of these studies, the rho transcription reporter method that uses the rho-specific SRE.L Luciferase was developed. This transcriptional reporter method described in detail in the Examples herein below was used for screening a small chemical library for possible rho inhibitors. Using this transcriptional method, a number inhibitors were identified as described below.

### B. Rho-Transcriptional Reporter and its Use in High Throughput Screening for Inhibitors of Rho-Mediated Signaling

Luciferase transcriptional readouts are useful for high throughput screening. Such assays employ an appropriate promoter that is ligated into a luciferase reporter vector adjacent to a luciferase reporter gene. Exemplary such vectors include, e.g., pGL3-basic (Promega, Madison Wis.). To examine luciferase activity, the luciferase reporter and other reporters are transfected into appropriate cells and luciferase assays are conducted in the presence and absence of the condition or compound being tested. Luciferase read-outs are determined using chemiluminescent reporter gene assay systems for the detection of luciferase.

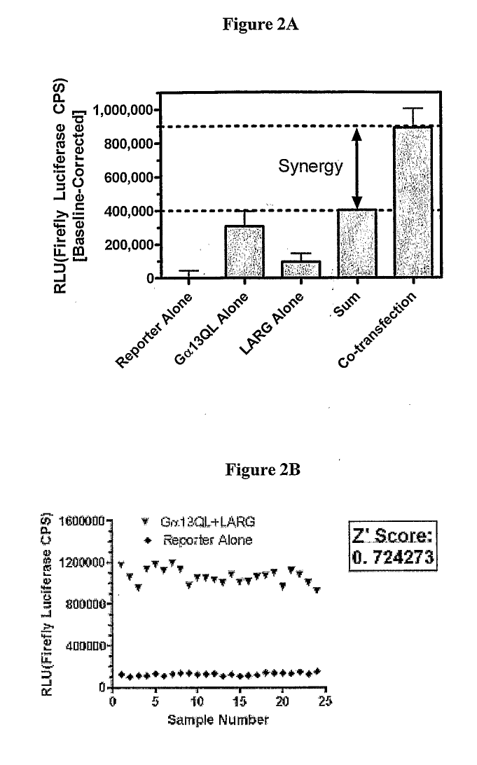

In exemplary embodiments of the present invention, HEK293T cells were transiently transfected with a 2×SRE.L-luciferase reporter plasmid that responds selectively to rho signaling (Hill et al., Cell 81:1159-1170, 1995). The SRE.L differs from a classical SRE in that it does not contain the TCF (Ternary Complex Factor) binding site (Miralles et al., Cell 113:329-342, 2003; Suzuki et al., Proc Natl Acad Sci USA 100:733-738, 2003; Hill et al., Cell 81:1159-1170, 1995). This expression plasmid is specific for rho/MKL/SRF driven gene expression. To stimulate rho, the upstream activators G13 and LARG (FIG. 1) were used. A constitutively active Gα13Q226L plasmid was co-transfected, along with an N-terminal myc-tagged LARG plasmid from Dr. Tohru Kozasa. Co-transfection of G13 and LARG together produced a synergistic response (as can be seen in FIG. 2 and (Suzuki et al., Proc Natl Acad Sci USA 100:733-738, 2003) which would permit detection of inhibitors working at G13, LARG, their interface, or anywhere downstream in the signaling pathway.

While in exemplary embodiments, the cells were co-transfected with G13 and LARG, it is contemplated that the cells may be co-transfected with any one or more activators from the rho-pathway. For example, the following table shows activators along the rho-signaling pathway in relation to the steps in the pathway that are being activated by the activator.

TABLE 1
Activators of the Rho-mediated pathway

| Step in pathway | Examples |
|---|---|
| Receptor (Group A) | LPA (LPA1, 2, 3, 4, 5), Thrombin (PAR1), muscarinic (m1, 3, 5), |
| | etc. (these are exemplary receptors whose activity is mediated |
| | through rho; other receptors also are contemplated and are known |
| | to those of skill in the art) |
| G protein (Group B) | G12 family (G12, G13), Gq family (Gq, G11, G14, G15, G16) or |
| | Activated mutants (QL mutants) of the same. |
| RhoGEF (Group C) | RGS RhoGEF (Leukemia-associated RhoGEF “LARG”, PDZ- |
| | rhoGEF, p115 rhoGEF), Gq-activated RhoGEFs (p63RhoGEF), |
| | etc. (it is noted that there are 213 rhoGEFs known and available to |
| | those of skill in the art. Any such rhoGEF may be used herein) |
| Rho (Group D) | RhoA, RhoC and their constitutively activated mutants (e.g. RhoA- |
| | GV, RhoC-GV) |
| Rho Effector | ROCK, mDia |
| (Group E) | |
| Co-activator | Megakaroycytic leukemia protein (MKL1, MKL2) |
| (Group F) | |
| Transcription factor | Serum response factor (SRF) |
| (Group G) | |

Preferably, the cells are co-transfected with activators of different steps along the rho-signaling pathway. For example, it is contemplated that screens may be set up in which the cells are co-transfected with one of the activators from Group A is combined with one activator from any of Groups B, C, D, E, F, or G in the above table (of course it should be understood that multiple activators from each of the groups may be used), other screens will use a combination of an activator from Group B, with an activator from any of Groups A, C, D, E, F, or G. Simply by way of example, contemplated co-transfections employ G13 as an exemplary member of Group B, and it is co-transfected with one or more other activators selected from the group consisting of LPA1, LPA2, LPA3, LPA4, LPA5, PAR1, muscarinic receptor m1, muscarinic receptor 3, muscarinic receptor 5, Leukemia-associated RhoGEF, PDZ-rhoGEF, p115rhoGEF, p63RhoGEF, RhoA and constitutively activated mutants thereof, RhoC and constitutively activated mutants thereof, ROCK, mDia, MKL1, MKL2, and SRF. In like manner, any of G12, Gq, G11, G14, G15, G16 or activated mutants (QL mutants) of G12, G13, Gq, G11, G14, G15, or G16 may be co-transfected with one or more other activators selected from the group consisting of LPA1, LPA2, LPA3, LPA4, LPA5, PAR1, muscarinic receptor m1, muscarinic receptor 3, muscarinic receptor 5, Leukemia-associated RhoGEF, PDZ-rhoGEF, p115rhoGEF, p63RhoGEF, RhoA and constitutively activated mutants thereof, RhoC and constitutively activated mutants thereof, ROCK, mDia, MKL1, MKL2, and SRF. It should be understood that all the combinations of the activators listed in the above table are expressly contemplated herein. The gene sequences for these activators are readily available through Genbank, EMBL and other publicly available gene databases, the sequences of which are incorporated herein by reference. An exemplary sequence of NcoI-myc epitope-BamHI-LARG is given in SEQ ID NO: 1. An exemplary sequence for human G-protein alpha 13 Q226L mutant is given in SEQ ID NO: 2.

In addition, while the preferred embodiments of the present invention employ HEK293 cells, it should be understood that any exemplary cell line may be employed for the methods of the present invention. Exemplary such cell lines include, but are not limited to, a Chinese hamster ovary (CHO) cell, a COS-7 cell, a LM(tk-) cell, a mouse embryonic fibroblast NIH-3T3 cell, a mouse Y1 cell, a 293 human embryonic kidney cell, including HEK293 and HEK293T cell, a Neuro-2A cell, a PC₁₂ cell, a SHSY-5Y cell, a HIT-T15 cell, a PC3 cell, a MDCK cell, a BHK cell, a NIH3T3 cell, a Swiss3T3 cell or a HeLa cell. Indeed, any host cell line that is typically employed in screening assays may be employed in the methods described herein and may even include insect cell lines such as, for example, Sf9, Sf21; amphibian cells such as Xenopus oocytes; assorted yeast strains; assorted bacterial cell strains; and others. Culture conditions for each of these cell types is specific and is known to those familiar with the art.

While the present invention uses the luciferase reporter, it should be understood that the cells can be transformed or transfected with any other nucleic acid molecule which performs a reporter function. Exemplary other such reporters include SEAP, green fluorescent protein, and so forth. It is well known that one of ordinary skill in the art can transform or transfect cells with expression vectors which require activation of, e.g., a receptor to cause the promoter to which the reporter molecule is operably linked, to function. Since activation of the receptor molecule depends upon ligand receptor interaction, one can determine the effect of a putative ligand or “anti-ligand” by measuring the reporter molecule function, and comparing it to a control.

Transient expression in a variety of mammalian, insect, amphibian, yeast, bacterial and other cells lines may be achieved using a variety of transfection methods including but not limited to: calcium phosphate-mediated, DEAE-dextran mediated; liposomal-mediated, viral-mediated, electroporation-mediated, and microinjection delivery. Each of these methods have been well documented in the art and may require optimization of assorted experimental parameters depending on the DNA, cell line, and the type of assay to be subsequently employed. In this regard, for a teaching of standard molecular biology techniques the skilled artisan may readily make reference to standard manuals or textbooks textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982); Sambrook et al., Molecular Cloning: A Laboratory Manual, 2nd Edition, Cold Spring Harbor Laboratory Press, New York (1989); Sambrook and Russell, Molecular Cloning: A Laboratory Manual, 3rd Edition, Cold Spring Harbor Laboratory Press, New York (2001); Ausubel et al., Current Protocols in Molecular Biology, John Wiley and Sons, New York (1992); and the various references cited therein. A number of eukaryotic expression vectors are known which allow multiple cloning sites for the insertion of one or more heterologous genes and their expression. Commercial suppliers include among others companies such as Stratagene, La Jolla, Calif., USA; Invitrogen, Carlsbad, Calif., USA; Promega, Madison, Wis., USA or BD Biosciences Clontech, Palo Alto, Calif., USA.

In the present invention, cells were transiently co-transfected with activators of the rho pathway and were used in high throughput screening assays. The present invention specifically contemplates methods of screening for other inhibitors of the rho-signaling pathway. In the present invention it was shown that co-transfection of a cell line with two activators of the rho-signaling pathway (LARG and G13) allowed the production of a cell line or population that facilitated the identification of inhibitors of the rho-signaling pathway. The inhibitors specifically identified herein inhibited rho-mediated gene transcription, SRF-stimulated gene transcription (an more particularly, MKL-dependent SRF-stimulated gene transcription) and inhibited key cancer cell functions such as LPA-stimulated DNA synthesis, cell proliferation, and matrix invasion. As such, these inhibitors can be used as therapeutic agents for any disorder that is mediated through LPA-stimulated DNA synthesis or rho-mediated gene transcription. The data presented herein show that the inhibitors have anti-cancer properties. This realization affords those of skill in the art ability to test various compounds for therapeutic activity that inhibits the activity of any combination of the activators of the rho pathway (rather than a single point). In one aspect, selected compounds will be those useful in treating cancer, metastatic growth or spread, cell proliferation, cell differentiation and the like. In another aspect, the inhibitors will be useful in the treatment of inflammatory disorders. The present section describes screening assays for identifying such compounds. In the screening assays of the present invention, the candidate substance may first be screened for basic biochemical activity—e.g., in vitro inhibition of a rho-mediated signal, and then tested for its ability to inhibit cell growth (e.g., growth of PC3 cells), at the cellular, tissue or whole animal level. To this effect, animal models of cancer and inflammatory diseases are well known to those of skill in the art (e.g., the nu/nu mouse and SCID mice have been employed in such in vivo testing of other therapeutic agents).

The present invention provides methods of screening for modulators of rho-mediated gene transcription using a luciferase based assay. It is contemplated that such screening techniques will prove useful in the identification of compounds that will inhibit, prevent, reduce, decrease, diminish or otherwise decrease gene transcription in a therapeutic manner and thus will be useful in the treatment of cancer and/or inflammatory diseases. In these embodiment, the present invention is directed to a method for determining the ability of a candidate substance to modulate rho-mediated gene transcription, generally including the steps of:

i) contacting a cell that is
a) transiently transfected with 2×SRE.L-luciferase reporter plasmid that responds selectively to rho signaling; and
b) is co-transfected with a first gene from Group A, B, C, D, E, F or G above, and a second different gene (and preferably from a different Group) from Group A, B, C, D, E, F or G above

ii) monitoring the chemiluminescent signal from said cell; and

iii) comparing the chemiluminescent signal in the presence and absence of said candidate substance; wherein an alteration in signal indicates that the substance is a modulator of rho-mediated gene transcription. Assays for determining chemiluminescence or other signal are well known to those of skill in the art. While in the above assay method the reporter is luciferase, it should be understood that other reporters, e.g., green fluorescent protein, SEAP and the like may be used.

To identify a candidate substance as a modulator in the assay above, one measures or determines the presence of the signal in the absence of the added candidate substance. One then adds the candidate substance to the cell and determines the response in the presence of the candidate substance. A candidate substance which changes the signal is indicative of a candidate substance having modulatory activity. In the in vivo screening assays, the compound is administered to a model animal, over period of time and in various dosages, and an alleviation of the symptoms associated with a rho-mediated disorder are monitored. Any improvement in one or more of these symptoms will be indicative of the candidate substance being a useful modulator. It is contemplated that the modulator is an inhibitor of rho-mediated activity.

The screening assays of the present invention have been modified to be performed as high throughput screens in the identification of new drugs.

As used herein the term “candidate substance” refers to any molecule that may potentially act as an inhibitor of a rho-mediated event. As discussed elsewhere in the present specification, the present invention has specifically identified agents of formula I that are useful inhibitors herein. In certain aspects, other substances may be identified as useful, such a candidate substance may be a protein or fragment thereof, a small molecule inhibitor, or even a nucleic acid molecule. Alternatively, useful pharmacological compounds will be compounds that are structurally related to other known modulators of rho-mediated activity. Specifically, it is contemplated that compounds of formula I will be used as targets of rational drug design in which each of the functional groups in formula I are modified and screened and compared to the activity of for example a compound of formula A or formula B.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds molded of active, but otherwise undesirable compounds.

Candidate compounds may include fragments or parts of naturally-occurring compounds or are found as active combinations of known compounds which are otherwise inactive. It is proposed that compounds isolated from natural sources, such as animals, bacteria, fungi, plant sources, including leaves and bark, and marine samples are assayed as candidates for the presence of potentially useful pharmaceutical agents. It will be understood that the pharmaceutical agents to be screened could also be derived or synthesized from chemical compositions or man-made compounds. In certain embodiments, the candidate substances are siRNA (also referred to as RNAi) molecules that are designed to inhibit the expression of one or more of the agents in Table 1. Example 1 herein below provides exemplary such siRNA molecules that are inhibitors of the rho-mediated pathway.

“Effective amounts” in certain circumstances are those amounts effective to reproducibly produce an alteration in a given rho-mediated activity, such as for example the level of expression or activity of a particular target in the rho pathway in comparison to their normal levels. Compounds that achieve significant appropriate changes in activity and/or expression of such a target will be used.

Significant changes in activity and/or expression will be those that are represented by alterations in activity of at least about 30%-40%, and in some aspects, by changes of at least about 50%, with higher values of course being possible.

The present invention particularly contemplates the use of various animal models for further testing the efficacy of the agents identified herein. Treatment of such animals with test compounds will involve the administration of the compound, in an appropriate form, to the animal. Administration will be by any route that could be utilized for clinical or non-clinical purposes, including but not limited to oral, nasal, buccal, or even topical. Alternatively, administration is by intratracheal instillation, bronchial instillation, intradermal, intratumoral subcutaneous, intramuscular, intraperitoneal or intravenous injection. Specifically contemplated are intratumoral instillation, inhalants and other mechanisms for delivery of the candidate substance locally to the site of a given disease.

Determining the effectiveness of a compound in vivo may involve a variety of different criteria. Such criteria include, but are not limited to, survival, reduction of tumor size, and improvement of general physical state including activity. It also is possible to perform histologic studies on tissues from these mice, or to examine the molecular and morphological state of the cells, which includes cell size, other morphological indicators or alteration in the expression of genes involved in the tested disorders.

### C. Inhibitors of Rho-Signaling

Using the above-described screening methods, the inventors identified novel inhibitors of the rho-signaling pathway. The compounds identified in the high throughput screen for rho inhibitors act at a distal site in gene transcription but have dramatic and specific effects on important cellular functions of the PC-3 prostate cancer cell. The potency (600-1000 nM) of these compounds, found in a relatively small screen, is remarkable. Furthermore, the correlation between the effects of the compounds on rho and on PC-3 cell functions suggests a tight link between rho function and cancer phenotype. The compound identified herein inhibit rho-stimulated gene transcription and PC-3 (cancerous) cell growth inhibition. It is believed that the compounds act primarily on MKL/SRF (directly and/or indirectly) to mediate the inhibitory effect.

In exemplary embodiments of the present invention, there is identified a class of rho inhibitors that has the general structural formula (I):

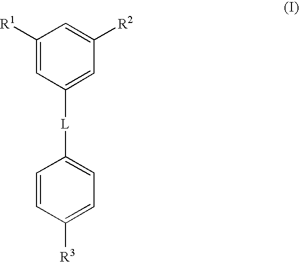

wherein R¹ and R², independently are selected from the group consisting of hydrogen, halo, C₁₋₃alkyl, C₁₋₃alkoxy, CF₃, OCF₃, C₁₋₂alkylOCH₂, NO₂, and CN, with the proviso that at least one of R¹ and R² is different from hydrogen;

R³ is selected from the group consisting of halo, C₁₋₃alkyl, C₁₋₃alkoxy, CF₃, OCF₃, C₁₋₂alkylOCH₂, NO₂, and CN: and

L is a linking group about four to about eight atoms or functional groups in length.

Typically, compound (I) has two terminal hydrophobic phenyl groups, and a hydrophilic linking group L. Accordingly, the atoms and functional groups of L can be, for example, C(═O), C(═S), NR^{a}, C═N(R^{a}), SO₂, SO, O, and S, wherein R^{a} is hydrogen, hydroxy, or methyl. The linking group L also can contain a phenyl ring or a C(R^{b})₂ group, wherein R^{b}, independently, is hydrogen or methyl. When the linking group L contains a phenyl ring, the phenyl is considered as contributing about two functional groups to the length of L.

In preferred embodiments, both R¹ and R² are different from hydrogen. In an especially preferred embodiment, both R¹ and R² are CF₃. In other preferred embodiments, R¹ is halo, i.e., fluoro, chloro, bromo, or iodo, and more preferably is chloro. In other preferred embodiments, the linking group L is about 5 to about 7 atoms and/or functional groups in length.

Preferred components and combinations comprising the linking group L include, but are not limited to, —NR^{a}—C(═O)—, —NR^{a}—SO₂—, —NR^{a}—O—, —C(R^{b})₂—C(═O)—, —O—C(—O)—, —N(R^{a})—N(R^{a})—, —O—C(R^{b})₂—, —SO₂—C₆H₄—, —NR^{a}—C₆H₄—, —C(═O)—C₆H₄—, —C(═S)—N(R^{a})—, —N(R^{a})—C(═NR^{a}))—, and —C(R^{b})₂—C₆H₄—.

Two non-limiting examples of Compound (I) are compounds (A) and (B):

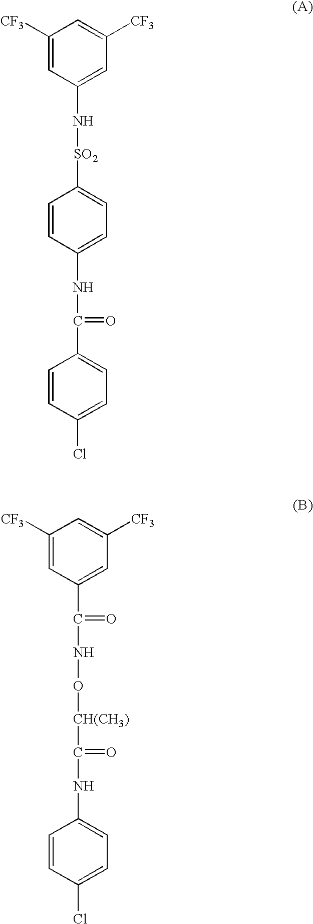

In compounds (A) and (B), the linking group is about six atoms or functional groups in length.

### D. Pharmaceutical Compositions and Methods of Treating Rho-Signaling Mediated Disorders

As can be seen from the data described in the Examples herein below, the methods of the present invention were used in a general HTS approach and identified structurally related compounds (CCG-1423 and CCG-977) that inhibit MKL/SRF-dependent gene transcription with IC₅₀'s of ˜800 nM and 3 μM, respectively. These inhibitors they strongly inhibit rho-stimulated gene transcription. In addition, these inhibitors inhibit key cancer cell functions such as LPA-stimulated DNA synthesis and spontaneous matrix invasion and are unique small molecule inhibitors of gene transcription.

These compounds are inhibiting MKL/SRF-dependent gene expression—thus they may have actions that are broader than just those on rho. For example, the inhibitors will be useful in the inhibition and/or treatment of any disorder that is related to MKL/SRF-dependent gene expression, including for example, cell proliferation, metastasis, inflammation and the like. The compounds of the present invention are useful in inhibiting cellular processes include cell survival, cell adhesion, cell migration, inducing proteolysis as it pertains to extracellular matrix remodeling, immune escape, angiogenesis and lymphangiogenesis, and target ‘homing.’ In particular embodiments, the compositions of the invention are useful in treating colon cancer, breast cancer, lung cancer, testicular germ cell cancer, and head and neck squamous-cell carcinoma. Other cancers also may be treated. In those embodiments where the compounds are used for the treatment of inflammatory disease, the compounds may be used to treat any type of such disease. Exemplary such diseases include, but are not limited to asthma, atopic dermatitis, allergic rhinitis, systemic anaphylaxis or hypersensitivity responses, drug allergies (e.g., to penicillin, cephalosporins), insect sting allergies and dermatoses such as dermatitis, eczema, atopic dermatitis, allergic contact dermatitis, urticaria, rheumatoid arthritis, osteoarthritis, inflammatory bowel disease e.g., such as ulcerative colitis, Crohn's disease, ileitis, Celiac disease, nontropical Sprue, enteritis, enteropathy associated with seronegative arthropathies, microscopic or collagenous colitis, eosinophilic gastroenteritis, or pouchitis resulting after proctocolectomy, and ileoanal anastomosis, disorders of the skin [e.g., psoriasis, erythema, pruritis, and acne], multiple sclerosis, systemic lupus erythematosus, myasthenia gravis, juvenile onset diabetes, glomerulonephritis and other nephritides, autoimmune thyroiditis, Behcet's disease and graft rejection (including allograft rejection or graft-versus-host disease), stroke, cardiac ischemia, mastitis (mammary gland), vaginitis, cholecystitis, cholangitis or pericholangitis (bile duct and surrounding tissue of the liver), chronic bronchitis, chronic sinusitis, chronic inflammatory diseases of the lung which result in interstitial fibrosis, such as interstitial lung diseases (ILD) (e.g., idiopathic pulmonary fibrosis, or ILD associated with rheumatoid arthritis, or other autoimmune conditions), hypersensitivity pneumonitis, collagen diseases, sarcoidosis, vasculitis (e.g., necrotizing, cutaneous, and hypersensitivity vasculitis), spondyloarthropathies, scleroderma, atherosclerosis, restenosis and myositis (including polymyositis, dermatomyositis), pancreatitis and insulin-dependent diabetes mellitus. Methods of treatment of any such diseases with the compounds of the invention are particularly contemplated.

The use of a screen where an entire pathway is screened rather than just a single molecular target has yielded an unexpected and novel set of compounds that can be used in methods of inhibiting rho-stimulated gene transcription, methods of inhibiting LPA-stimulated DNA synthesis, inhibition of MKL/SRF-dependent gene expression. Further the compounds will be useful in therapeutic compositions for the treatment of cancer, metastases and inflammatory disorders. In specific embodiments, the compounds of the present invention are identified in a mammalian cell-based high throughput assay for identifying modulators of a rho-mediated activity comprising contacting a host cell that has been transiently transfected with reporter plasmid that responds selectively to rho signaling expressing and has been further co-transfected with a first and a second activator of the rho-signaling pathway with a candidate modulator of rho; monitoring the signal produced from said cell; and comparing the signal in the presence and absence of said candidate substance; wherein an alteration in signal indicates that the substance is a modulator of rho-mediated gene transcription.

In a specific screen, the reporter plasmid is a bioluminescent reporter plasmid, such as for example, 2×SRE.L-luciferase reporter plasmid. Typical screens can be set up in which the first activator of rho-signaling pathway is selected from the group consisting of one of the activators from Group A, B, C, D, E, F and G in Table 1. In such screens, the second activator of rho-signaling pathway is selected from the group consisting of one of the activators from Group A, Be C, D, E, F and G in Table 1, and said second activator is different from said first activator. Preferably, the second activator of rho-signaling pathway is selected from the group consisting of one of the activators from Group A, B, C, D, E, F and G in Table 1 and said second activator is from a different Group than the first activator. Thus, for example, the first activator is selected from the group consisting of LPA1, LPA2, LPA3, LPA4, LPA5, PAR1, muscarinic receptor m1, muscarinic receptor 3, muscarinic receptor 5, Leukemia-associated RhoGEF, PDZ-rhoGEF, p115rhoGEF, p63RhoGEF, RhoA and constitutively activated mutants thereof, RhoC and constitutively activated mutants thereof, ROCK, mDia, MKL1, MKL2, SRF, G12, G13, Gq, G11, G14, G15, G16, QL mutant of G12, QL mutant of G13, QL mutant of Gq, QL mutant of G11, QL mutant of G14, QL mutant of G15, and QL mutant of G16 and the second activator is selected from the group consisting of LPA1, LPA2, LPA3, LPA4, LPA5, PAR1, muscarinic receptor m1, muscarinic receptor 3, muscarinic receptor 5, Leukemia-associated RhoGEF, PDZ-rhoGEF, p115rhoGEF, p63RhoGEF, RhoA and constitutively activated mutants thereof, RhoC and constitutively activated mutants thereof, ROCK, mDia, MKL1, MKL2, SRF, G12, G13, Gq, G11, G14, G15, G16, QL mutant of G12, QL mutant of G13, QL mutant of Gq, QL mutant of G11, QL mutant of G14, QL mutant of G15, and QL mutant of G 16, wherein said second activator is different from said first activator.

Using such screens, the inventors identified the novel compounds of the present invention, and determined that the novel compounds have a general structural formula (I):

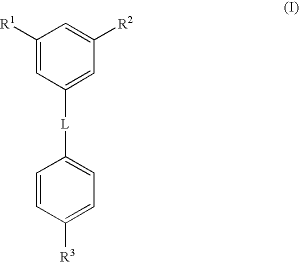

wherein R¹ and R², independently are selected from the group consisting of hydrogen, halo, C₁₋₃alkyl, C₁₋₃alkoxy, CF₃, OCF₃, C₁₋₂allylOCH₂, NO₂, and CN, with the proviso that at least one of R¹ and R² is different from hydrogen;

R³ is selected from the group consisting of halo, C₁₋₃alkyl, C₁₋₃alkoxy, CF₃, OCF₃, C₁₋₂alkylOCH₂, NO₂, and CN: and

L is a linking group about four to about eight atoms or functional groups in length. In preferred such compounds, the general formula I comprises two terminal hydrophobic phenyl groups, and a hydrophilic linking group L. More preferably, L is selected from the group consisting of C(═O), C(═S), NR^{a}, C═N(R^{a}), SO₂, SO, O, S, a phenyl ring and C(R^{b})₂ group, wherein R^{a} independently, is hydrogen, hydroxy, or methyl and wherein R^{b}, independently, is hydrogen or methyl.

In specific and particular embodiments, exemplary compounds of formula I are isolated compounds that have formula A or formula B:

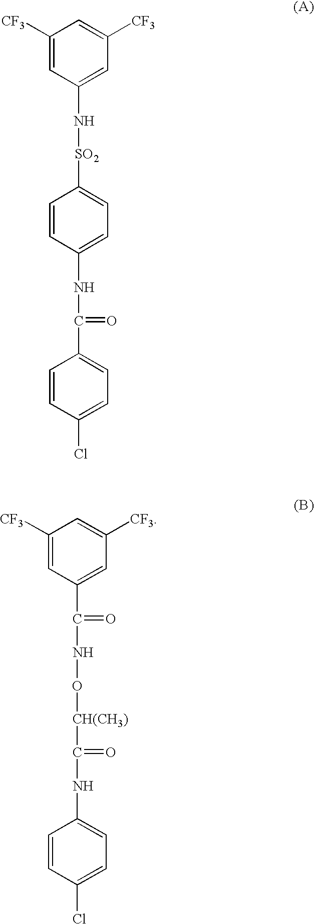

The assays are set up such that the host cell is selected from the group consisting of MDCK, HEK293, HEK293 T, 1321N1, BHK, COS, NIH3T3, Swiss3T3 and CHO. In specific embodiments, the cell is an HEK293 cell. Preferably, the cells are seeded onto a well of a multi-well test plate. The invention therefore uses screening assays that use a cell in high throughput screening for modulators of rho-mediated activity, wherein said cell has been transiently or stably transfected 2×SRE.L-luciferase reporter plasmid that responds selectively to rho signaling expressing and has been further co-transfected with a first and a second activator of the rho-signaling pathway with a candidate modulator of rho.

Therapeutic compositions that comprise one or more compounds of the general formula I are particularly preferred. The pharmaceutical compositions also may include another agent that is used for the treatment of a disorders being treated by the compounds of the general formula (I). As such, in general terms combination therapy is specifically contemplated. Combination therapy with anticancer therapeutic agents or with anti-inflammatory agents is particularly contemplated. Each of these preparations is in some aspects provided in a pharmaceutically acceptable form optionally combined with a pharmaceutically acceptable carrier. These compositions are administered by any methods that achieve their intended purposes. Individualized amounts and regimens for the administration of the compositions for achieving a therapeutic effect will be determined readily by those with ordinary skill in the art using assays that are used for the diagnosis of the disorder and determining the level of effect a given therapeutic intervention produces.

Compositions within the scope of this invention include all compositions comprising at least one compound described herein above in an amount effective to achieve its intended purpose of inhibiting, reducing, preventing, abrogating or otherwise producing a decrease in an SRF- and/or rho-mediated signal in a given pathway. In some aspects, such treatment will result in an alleviation of one or more symptoms of cancer, metastatic growth, or general growth of a cancer cell.

It is understood that the suitable dose of a composition according to the present invention will depend upon the age, health and weight of the recipient, kind of concurrent treatment, if any, frequency of treatment, and the nature of the effect desired. However, the dosage is tailored to the individual subject, as is understood and determinable by one of skill in the art, without undue experimentation. This typically involves adjustment of a standard dose, e.g., reduction of the dose if the patient has a low body weight.

The total dose of therapeutic agent may be administered in multiple doses or in a single dose. In certain embodiments, the compositions are administered alone, in other embodiments the compositions are administered in conjunction with other therapeutics directed to the disease or directed to other symptoms thereof.

In some aspects, the compositions of the invention are formulated into suitable pharmaceutical compositions, i.e., in a form appropriate for in vivo applications in the therapeutic intervention of a specific disorder that involves rho and/or SRF-stimulated gene transcription. Generally, this will entail preparing compositions that are essentially free of pyrogens, as well as other impurities that could be harmful to humans or animals. These formulations can be in the form of an oral medicament or can be formulated for another routes of administration (e.g. injection and the like). The compositions may ideally be formulated to be administered locally, for example, directly into a tumor site or a site of inflammation. Receptor-mediated uptake into the site of interest is especially useful. Systemic delivery of the compositions also is specifically contemplated.

One will generally desire to employ appropriate salts and buffers to render the compositions stable and allow for uptake of the compositions at the target site. Generally the protein compositions of the invention are provided in lyophilized form to be reconstituted prior to administration. Alternatively, the compositions of general formula I are likely formulated into tablet form. Buffers and solutions for the reconstitution of the therapeutic agents may be provided along with the pharmaceutical formulation to produce aqueous compositions of the present invention for administration. Such aqueous compositions will comprise an effective amount of each of the therapeutic agents being used, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also are incorporated into the compositions.

Methods of formulating compounds for therapeutic administration also are known to those of skill in the art. Administration of these compositions according to the present invention will be via any common route so long as the target tissue is available via that route. However, other conventional routes of administration, e.g., by subcutaneous, intravenous, intradermal, intramuscular, intramammary, intraperitoneal, intrathecal, intraocular, retrobulbar, intrapulmonary (e.g., term release), aerosol, sublingual, nasal, anal, vaginal, or transdermal delivery, or by surgical implantation at a particular site also is used particularly when oral administration is problematic. The treatment may consist of a single dose or a plurality of doses over a period of time.

In certain embodiments, the active compounds are prepared for administration as solutions of free base or pharmacologically acceptable salts in water suitably mixed with a surfactant, such as hydroxypropylcellulose. Dispersions also are prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. In some aspects, the carrier is a solvent or dispersion medium containing, for example, water, ethanol, polyol (for example, glycerol, propylene glycol, and liquid polyethylene glycol, and the like), suitable mixtures thereof, and vegetable oils. The proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions is brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also are incorporated into the compositions.

In some aspects, the compositions of the present invention are formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also are derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. In certain embodiment, parenteral administration of the therapeutic compounds is carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose is calculated according to body weight, body surface areas or organ size. The availability of animal models is particularly useful in facilitating a determination of appropriate dosages of a given therapeutic. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Typically, appropriate dosages are ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention are useful in fields of human medicine and veterinary medicine. Thus the subject to be treated is a mammal, such as a human or other mammalian animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

The present invention also contemplated kits for use in the treatment of disorders that involve rho-signaling. Such kits include at least a first composition comprising as an active ingredient a compound of formula (I) (more specifically, formula A and/or formula B) described above in a pharmaceutically acceptable carrier. Another component is a second therapeutic agent for the treatment of the disorder along with suitable container and vehicles for administrations of the therapeutic compositions. As noted above, combination therapy with the compounds of the present invention and other convention chemotherapeutic agents is specifically contemplated. Conventional chemotherapeutic agents suitable for use in such combination therapies maybe any known pharmaceutically acceptable agent that depends, at least in part, on interfering with cellular structure and/or metabolism for its anticancer activity. Examples of conventional chemotherapeutic agents include, but not limited to, platinum compounds such as cisplatin, carboplatin and their analogs and derivatives; alkylating agents such as chlorambucil, nitrogen mustards, nitromin, cyclophosphamide, 4-hydroperoxycyclophosphamide; 2-hexenopyranoside of aldophosphamide, melphalan, BCNU, CCNU, methyl-CCNU, uracil mustard, mannomustine, triethylenemelamine, chlorozotocin, ACNU, GANU, MCNU, TA-77, hexamethylmelamine, dibromomannitol, pipobroman, epoxypropidine, epoxypiperazine, ethoglucide, piposulfan, dimethylmilelane, bubulfan, inprocuon, threnimone, thio-TEPA and Aza-TEPA; antimetabolites such as 5-fluorouracil, folic acid, methotrexate (MTX), 6-mercaptopurine, aminopterin, 8-azaguanine, azathioprine, uracil, cytarabine, azaserine, tegaful, BHAC SM108, cytosine arabinoside, cispuracham, diazamycine, HCFU, 5′DFUR, TK-177 and cyclotidine; plant components such as cyclophosphamide, 4-hydroperoxycyclophosphamide, thiotepa, taxol and related compounds, doxorubicin, daunorubicin and neocarzinostain; antibiotics such as bleomycin, daunomycin, cyclomycin, actinomycin D, mitomycin C, carzinophylin, macrocinomycin, neothramycin, macromomycin, nogaromycin, cromomycin, 7-o-methylnogallol-4′-epiadriamycin,4-demethoxydaunorubicin, streptozotocin DON and mitozanthron; bis-chloroethylating agents, such as mafosfamide, nitrogen mustard, nornitrogen mustard, melphalan, chlorambucil; hormones such as estrogens; bioreductive agents such as mitomycin C and others such as mitoxantrone, procarbazine, adriblastin, epirubicin, prednimustine, ifosfamid, P-glycoprotein inhibitors such as thaliblastine and protein kinase inhibitors such as protein kinase C inhibitor (ilmofosine). Chemotherapeutic agents particularly refer to the antimicrotubule agents or tubulin targeting agents including vinca alkaloids; vinca alkaloids such as etoposide, podophyllotoxin, vincristine and vinblastine; taxanes (paclitaxel, docetaxel and precursor taxane (10-deacetylbaccatin III), arsenic salts, colchicin (e), thio-colchicine, colchiceine, colchisal and other colchium salts; epipodophyllotoxins (etoposide), cytochalasins (such as A-E, H, J), okadaic acid, carbaryl and it's metabolites such as naphthol or naphthyl compounds including 1-naphthol, 2-naphthol, 1-naphthylphosphate, malonate, nocodazole (methyl-(5-[2-thienyl-carbonyl]-1H-benzimidazol-2-yl)carbamate), cryptophycin (CP) and its analogues such as CP-52, wortmannin, 12-0-tetradecanoylphorbol-13-acetate (TPA), 14-3-3 sigma and its homologs (such as rad24 and rad25), Ustiloxin F, monocrotalines such as monocrotaline pyrrole (MCTP), estramustine and the inhibiting agents of adenosine. These chemotherapeutic agents may be used either alone or in combination. Preferably, one antimetabolite and one antimicrotubule agent are combined, and more preferably, taxol, cisplatin, chlorambucil, cyclophosphamide, bleomycin, or 5-fluorouracil which have different tumor killing mechanisms are combined. The combination containing arsenic compounds, colchicin, colchicine, colchiceine, colchisal, colchium salts, vinblastine, paclitaxel and related compounds that interfere with the cytoskeletons are most preferred. As new chemotherapeutic agents and drugs are identified and become available to the art, they may be directly applied to the practice of the present invention. The kits may additionally comprise solutions or buffers for effecting the delivery of the first and second compositions. The kits may further comprise additional compositions which contain further inhibitors of cancer growth, inflammation and the like. The kits may further comprise catheters, syringes or other delivering devices for the delivery of one or more of the compositions used in the methods of the invention. The kits may further comprise instructions containing administration protocols for the therapeutic regimens.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus are considered to constitute certain aspects for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Materials—The three myc-tagged human RGS-rhoGEF plasmids in pcDNAmyc and the SRE.L Luciferase rho reporter construct were described previously (Suzuki, et al., Proc. Natl. Acad. Sci. U.S.A. 100, 733-738, 2003). The control Renilla luciferase construct, pRL-TK, was purchased from Promega. The C3 exotoxin expression construct in pcDNA3.1.

siRNA design and synthesis has been described in detail (Wang et al., Methods Enzymol. 389). Briefly, 21-nucleotide synthetic RNA with the following sequences and their complements (plus 3′ TT overhangs on each) were synthesized by the Qiagen siRNA synthesis service (Xenogene Inc.) and high performance liquid chromatography-purified. The sequence targeted was in the RGS domain for all three RGS-rhoGEFs: p115rhoGEF (CATACCATCTCTACCGACG) (SEQ ID NO: 3), PDZrhoGEF (ACTGAAGTCTCGGCCAGCT) (SEQ ID NO: 4), and LARG (GAAACTCGTCGCATCTTCC) (SEQ ID NO: 5). Duplexes (with 3′ TT overhangs) were prepared by annealing sense and antisense strands to form double-stranded RNA. The oligonucleotide pairs were resuspended at 0.3 mg/ml (total) in buffer containing 100 mM potassium acetate, 30 mM HEPES-KOH, 2 mM magnesium acetate, pH 7.4, and heated to 90° C. for 1 min and then incubated at 37° C. for 1 h. Aliquots were then placed in small vials to make siRNA stock solutions at 300 ng/μl or about 20 μM and frozen at −20° C.

LARG, p115-rhoGEF, and G antibodies were purchased from Santa Cruz Biotechnology Inc. (N-14, catalog number sc-15439; C-19, catalog number sc-8492, T-20, catalog number sc-378). Affinity-purified polyclonal anti-PDZ-rhoGEF was generated with a synthetic peptide from rat PDZ-rhoGEF (GTRAP48, KTPERTSPSHHRQPSD) as described previously (Jackson et al., Nature 410, 89-93, 2001). Secondary antibodies were bovine anti-goat IgG-horseradish peroxidase (catalog number sc-2352) and goat anti-rabbit IgG-horseradish peroxidase (catalog number sc-2054). The PAR1 agonist peptide SFLLRN was from Bachem (catalog number H-8365, King of Prussia, Pa.). HEK293 and PC-3 cell lines are readily available from commercial sources.

Transfection of siRNAs into Cells—For Western blot analysis, HEK293 cells were transiently transfected in a 6-well plate with 2 μg/well of either active rhoGEF siRNAs (LARG, PDZ, p115) or mutant inverted siRNAs and 8 μl of LipofectAMINE 2000. After 72 h, protein lysates were prepared as described (Wang et al., J. Biol. Chem. 277, 24949-24958). For luciferase assays in 96-well plates, one rhoGEF plasmid DNA (p 115 (2 ng), PDZ (5 ng), or LARG (5 ng)) was introduced into HEK293 cells with 30 ng of rhoGEF siRNAs or inverted mutant siRNAs with 0.2 μl of LipofectAMINE 2000 together with the dual luciferase reporters (SRE.L and pRL-TK at 3 ng and 30 ng/well, respectively).

Western Blot Analysis—Western blot analysis is done with RGS-rhoGEF-specific antibodies and ECL detection as described previously (Wang et al., J. Biol. Chem. 277, 24949-24958). The same polyvinylidene difluoride membrane was stripped and re-blotted with anti-G protein ^{β}subunit antibody as loading control. Blots were quantitated as described previously (Wang et al., J. Biol. Chem. 277, 24949-24958).

Luciferase Assays—Twenty-four hours post-transfection with firefly and Renilla vectors, HEK293 cells were serum-starved (0.5% serum), and then at 48 h, luciferase activity was determined using the dual luciferase assay kit (Promega, Madison, Wis.) according to the manufacturer's instructions. The ratio of firefly to Renilla luciferase counts was calculated. Data are expressed as the percent of the control value (samples without siRNA).

GST-rhotekin Pull-down Assay—siRNA (si) or mutant controls (inv) were introduced (10 μg/10-mm dish) into HEK293 cells with 30 μl of LipofectAMINE 2000 in duplicate 100-mm dishes. Thirty-six hours after transfection, cells were switched to medium with 0.5% serum, then 18 h later stimulated with 300 nM thrombin for 5 min. Cells from each dish were lysed in 0.5 ml of lysis buffer containing 50 nm Tris, pH 7.5, 10 mM MgCl₂, 0.5 M NaCl, 2% IGEPAL, and 5% sucrose. The active rho was precipitated with GST-rhotekin beads (Cytoskeleton Inc., Denver, Colo.). Western blots with anti-rhoA antibody were done to assess the amount of active rhoA. Aliquots of total lysate were also analyzed for the amount of rho present.

Cell Rounding Assay—The day before transfection, PC-3 cells were grown on laminin-coated coverslips in 12-well plates until −80% confluent. Cells were then transfected with 0.2 μg of EGFP cDNA. To disrupt rho signaling GFP was co-transfected with either a C3 exotoxin expression vector (0.5 μg) or with 2.0 μg of the indicated siRNA or inverted control along with 8 μl/well of LipofectAMINE 2000. At 45 h post-transfection, cultures were changed to serum-free medium and 6 h later treated with either buffer, thrombin (100 nM), or LPA (50 μM) for 30 min. Cells were then fixed with 4% paraformaldehyde for 10 min and GFP images obtained using an Olympus fluorescence microscope with a 20× objective. Fluorescent cells were counted (100-300 per coverslip) and the percentage of rounded cells determined. Cell rounding at 30 min was dose-dependent (EC₅₀ 80 nM for thrombin and 50 μM for LPA) and reversible upon removal of thrombin for 45 min.

### Example 2

### Demonstration of Receptor-Specific Activation of RGS-rhoGEFs

All three RGS-rhoGEFs are expressed in HEK293 cells as detected by both reverse transcritase-PCR analysis (Wang et al. Methods Enzymol. 389) and Western blot analysis. In the latter analyses, HEK293T cells were transiently transfected with active rhoGEF siRNAs against LARG, PDZrhoGEF, or p115rhoGEF (si) or the related mutant inverted siRNAs (inv) and protein lysates prepared at 72 h. Western blot analysis with RGS-rhoGEF-specific antibodies or a G^{β} subunit loading control were performed and revealed suppression of endogenus rhoGEF proteins by the siRNAs.

HEK293 cells also exhibit a substantial thrombin-stimulated rho activation. In these analyses, LARG siRNA or its mutant siRNA (LARGinv) were introduced (10 μg/dish) into HEK293T cells and thrombin-stimulated rho activation measured as described under Example 1. Cells were treated with (+) or without (−) thrombin (300 nM for 5 min). The active rho was precipitated with GST-rhotekin beads, and Western blots with anti-rhoA antibody were done to assess the amount of active rhoA.

To assess the role of RGS-rhoGEFs in receptor signaling, a series of synthetic oligo-siRNAs targeted against the RGS domain of the three human rhoGEFs were designed. Two oligonucleotides each against LARG and PDZrhoGEF and 5 against p115rhoGEF were analyzed (Wang et al., Methods Enzymol. 389), and the most active and specific three siRNAs were used in this study. They suppress the expression of their cognate rhoGEF but do not affect either G^{β} used as a loading control or the other rhoGEFs. Their specificity was also demonstrated in a functional effect on rho-mediated gene expression. A modified SRE.L luciferase reporter construct (Suzuki et al., Proc. Natl. Acad. Sci. U.S.A. 100, 733-738, 2003), which responds to serum response factor-megakaryocytic acute leukemia transcription complexes but not serum response factor-ternary complex factor complexes was used and provided a rho-dependent transcription response. Luciferase expression is strongly enhanced by transfected wild-type G_{α13} and the constitutively active G_{α13}QL mutant (19- and 54-fold over reporter alone, respectively), and by transfection of all three of the RGS-rhoGEFs. In these studies, each rhoGEF plasmid DNA (p115, PDZ, or LARG) was introduced into HEK293T cells using LipofectAMINE 2000 in a 96-well plate along with the dual luciferase reporters (SRE.L and pRL-TK) and a panel of different rhoGEF siRNAs (+) or inverted mutant siRNAs (−). Firefly and Renilla luciferase activities were measured 48 h post-transfection as described in Example 1 and the ratio of firefly to Renilla luciferase counts calculated. The reporters alone gave ratios of 25±3 and each rhoGEF stimulated firefly expression more than 10-fold. Firefly/Renilla ratios for the siRNAs (+ and −) were expressed as a percent of that for buffer controls with only the rhoGEF and reporter cDNAs transfected.

In each case, firefly luciferase expression is reduced to base line by co-transfecting C3 exotoxin consistent with the literature showing that G₁₃- and rhoGEF-stimulated gene expression is rho-dependent. The rhoGEF-stimulated luciferase signal is also completely eliminated by 0.5 μM latrunculin B. Co-transfection of the siRNAs targeting LARG, p115rhoGEF, or PDZrhoGEF strongly and specifically reduced the luciferase response to the targeted RGS-rhoGEF but had minimal effects on luciferase expression stimulated by the other two RGS-rhoGEFs. As an additional control for specificity, an inverted control siRNA (−) for each of the active siRNAs (+) was included and had minimal effects. The incomplete inhibition of the luciferase response by the siRNAs may be due to: 1) a strongly amplified signaling cascade which is integrated over 48 h, 2) some maintained expression of RGS-rhoGEF in the presence of siRNA, or 3) incomplete overlap of transfection of the siRNA and the RGS-rhoGEF plasmid. The latter mechanisms seems unlikely given the nearly 90% transfection efficiency of these cells with a GFP reporter plasmid plus the virtually complete suppression of endogenous protein for LARG and p115rhoGEF. The data, however, indicate a substantial and specific suppression of RGS-rhoGEFs by these transfected siRNAs.

To assess the role of the RGS-rhoGEFs in receptor-mediated signaling, we used the thrombin-stimulated activation of rho as detected by precipitation of GTP-bound active rhoA with the effector domain fusion, GST-rhotekin. Thrombin stimulates rho activation in HEK293 cells probably via an endogenous PAR1 receptor (Hollenberg et al., Can. J. Physiol. Pharmacol. 75, 832-841, 1997). Transfection of HE 93 cells with the active LARG siRNA (si) shows an essentially complete block of thrombin-stimulated rho activation. While this assay exhibits significant variability, only the LARG siRNA reduced the thrombin-stimulated rho activation significantly. To assess signaling by other receptors, we attempted similar measurements with LPA. While there was some stimulation of rhoA activation by LPA in HEK293 cells, it was substantially smaller than that induced by thrombin and not sufficient to permit analysis with the siRNAs. Thus the inventors turned to a different cell line and a different rho response.

rho stimulates several types of cytoskeletal rearrangements. Stress fiber formation in NIH or Swiss 3T3 cells is a classic rho response (Ridley and Hall, Cell 70, 389-399, 1992). In other cell types such as the 1321N1 astrocytoma line, cell rounding is observed as a thrombin-stimulated G_{12/13}-mediated rho response (Majumdar et al., J. Biol. Chem. 274, 26815-26821, 1999). A similar cell rounding response occurs in the human prostate cancer cell line, PC-3. After thrombin stimulation, there was some appearance of stress fibers following phalloidin staining, but the more prominent effect was cell rounding. In these studies, at 48 h after transfection, cells were stimulated with thrombin (100 n_{M}) or LPA (50 μ_{M}) for 30 min, fixed, and fluorescence images taken. Co-transfection of C3 toxin abolished both thrombin- and LPA-induced cell rounding indicating the involvement of rhoA. B, siRNA effect on agonist-stimulated cell rounding. The rhoGEF siRNAs (+) or mutant inverted siRNAs (−) were introduced into PC-3 cells together with pEGFP plasmid DNA. At 48 h post-transfection cells were stimulated with thrombin or LPA and analyzed as described above. One-hundred green cells from each coverslip were counted, and the percentage of round cells was determined. The PAR1 agonist peptide SFLLRN (100 μM) was used to stimulate PC-3 cell rounding after treatment with LARG or PDZrhoGEF siRNA and it was shown that PAR1 receptor mediates LARG-dependent cell rounding in PC-3 cells. Only LARG siRNA inhibited PAR1 agonist peptide effects.

Since the transfection efficiency of the PC-3 cells (30-50%) was lower than that in HEK293, co-transfected GFP reporter plasmid was used to permit the selective assessment of the transfected cell population. The cell rounding response to thrombin was reduced by 80-90% by co-transfecting a C3 exotoxin expression plasmid with the GFP reporter. The basal cell shape, however, was not affected by C3 toxin. In addition to thrombin responses, LPA also stimulated PC-3 cell rounding in a rho-dependent manner.

The thrombin and LPA responses in PC-3 cells were then tested with the RGS-rhoGEF siRNAs which on Western blots show suppression of the appropriate rhoGEF (data not shown). As expected from the HEI293 data, the LARG siRNA nearly completely abolished the thrombin-stimulated PC-3 cell rounding response. This is mediated by the PAR1 type of thrombin receptor, since cell rounding stimulated by the PAR1 agonist peptide SFLLRN was also inhibited by LARG siRNA. The negative control inverted LARG siRNA (−) did not affect thrombin-stimulated cell rounding. Also, the p115rhoGEF and PDZrhoGEF siRNAs did not inhibit the thrombin response indicating that LARG represents the primary downstream pathway from PAR1 to rho activation in PC-3 cells. Surprisingly, the LARG siRNA did not inhibit cell rounding induced by LPA but the PDZrhoGEF siRNA did. This shows that the LPA receptor uses a different RGS-rhoGEF to induce rho responses. It is known that PC-3 cells express LPA₁ and LPA₂ but not LPA₃ receptors (Xie et al. J. Biol. Chem. 277, 32516-32526, 2002). The LPA data also provide additional controls; the LARG siRNA effect on thrombin responses is specific for thrombin and not other rho-activating stimuli, and the lack of effect of PDZrhoGEF siRNA on thrombin responses is not due to incomplete knockdown of the PDZrhoGEF protein as the siRNA was able to disrupt the LPA response.

The above data provide the first direct demonstration that RGS-rhoGEFs mediate GPCR signaling to rho as well as showing receptor specificity in the use of RGS-rhoGEFs with PAR1 using LARG in both HEK293 and PC-3 cells and the LPA receptor using PDZrhoGEF in PC-3 prostate cancer cells. Since thrombin stimulates proliferation and vascular endothelial growth factor secretion of prostate cancer cells and prostate cancer metastatic to bone has increased levels of PAR1, the ability to block receptor-stimulated rho signaling could represent an important approach to regulating cancer cell growth and metastasis.

### Example 3

### Demonstration of High-Throughput Screening for G13/LARG rho Pathway Inhibitors

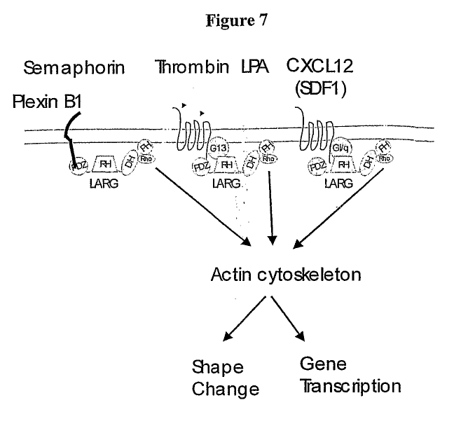

Example 2 shows analyses designed to determine the of the role of RGS-rhoGEFs in rho signaling by different receptors (see FIG. 7) using synthetic RNAi against the three members of this protein family. The data in Example 2 demonstrate that in PC-3 cells, the thrombin receptor (PAR1) utilized LARG while the LPA receptor utilized PDZ-rhoGEF for inducing cell rounding. In addition, direct measurements of thrombin-induced rho activation in HEK293T cells (using GST-rhotekin pulldown) also showed a dependence on LARG. In Example 2, the rho transcription reporter method that uses the rho-specific SRE.L Luciferase was developed. The present example uses the reporter to identify rho inhibitors.

To use the reporter assay readout for high throughput chemical inhibition studies, the assay was modified to accommodate chemical inhibitors. Transfection was performed in 10 cm dishes with Lipofectamine 2000. Five hours after transfection, the cells were trypsinized and 30,000 cells per well were transferred into sterile white flat-bottom 96-well plates pre-spotted with 1 μl of chemical compound in DMSO (10 μM final conc). Cells were grown overnight in low serum (0.5% FBS/DMEM medium) exposed to the chemical compounds (in 1% DMSO) for approximately 18 hours before luminescence was read with Steady-Glo luciferase reagent (Promega) in a Victor II plate reader. As a positive inhibitor control, 0.5 μM of Latrunculin B was added to 8 wells per plate. In addition, 8 wells were treated with DMSO only. Latrunculin B is a marine toxin from the Red Sea sponge that is known to sequester monomeric G-actin in the cell and block rho-mediated transcriptional events (Miralles et al., Cell 113:329-342, 2003). The percent inhibition of the Gα13 QL/LARG stimulated SRE-luciferase response was calculated by setting the mean of the negative control (DMSO only) at 0% and the mean of the positive control (Latrunculin B) at 100%. The Z′ score of the assay for high throughput screening was excellent at 0.7 (FIG. 2) indicating reproducibility well-suited to HTS studies.

An exemplary screen for small molecule inhibitors of the Gα13/LARG/SRE transcriptional signaling pathway utilized a 2000 compound subset of the Maybridge Diverse Chemical Compound Library at 10 μM in the firefly luciferase assay described above. SRE transcription was stimulated by co-transfection of the constitutively active Gα₁₃QL with LARG. Table 2 shows outlines the results from that small screen. A cutoff of 75% inhibition was chosen to identify hits for inhibition of the Gα13/LARG/SRE transcriptional signaling pathway. A total of 39 out of 2000 compounds met this criterion. One problem with this “loss of function screen” is that many compounds could be toxic or could non-specifically inhibit cell function. To address this issue, a dual luciferase method with a “flash” luciferase substrate plus CMV-Renilla was used as a non-SRF-dependent reporter to eliminate toxic compounds (Table 2). This screen yielded only 4 compounds that were active and specific for the SRE.L reporter signal. These 4 compounds were tested against luciferase itself, which revealed that two of the 4 were direct luciferase inhibitors, leaving 2 specific inhibitors of the rho pathway (0.1% “hit rate”).

TABLE 2
High Throughput Screening for
G13/LARG rho pathway inhibitors

| | |
|---|---|
| Total # of Compounds Screened: | 2000 |
| Positive Hits from Primary Screen | 39 |
| (Steady-Glo Luciferase Assay) | |
| Secondary Screen/Dose Response | 20 - Didn't inhibit SRE- |
| (Dual Flash Luciferase Assay) | Luciferase |
| | 5 - Inhibited Renilla |
| | 14 - True Positives |
| Available for Re-Supply | 13 |
| Secondary Screen/Dose Response Redo | 1 - Didn't inhibit SRE- |
| (Dual Flash Luciferase Assay) | Luciferase |
| | 8 - Inhibited Renilla |
| | 4 - True Positives |
| Direct Firefly Luciferase Inhibition | 2 - Direct inhibitors of |
| | firefly luciferase |

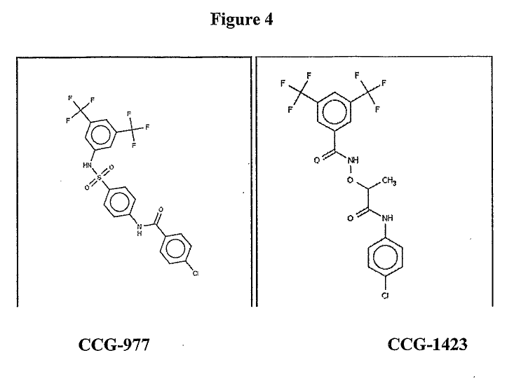

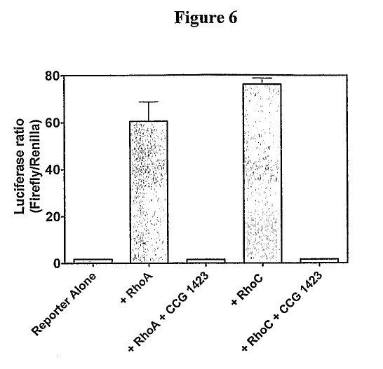

The two compounds with specific effects on G13/LARG signaling are shown in FIG. 4. Surprisingly, these inhibitors share very similar structures with a meta-bis-trifluoromethyl benzene at one end and a chlorphenyl at the other. The compounds CCG-1423 and CCG-997 were tested in dose response curves and had IC50 values of ˜1000 and 3000 nM, respectively in three different experiments (FIG. 4 for CCG-1423). Interestingly, this signal is only partially blocked by the rho kinase inhibitor (Y-27632) though luciferase expression stimulated by transfecting in the kinase ROCK was fully inhibited by Y-27632. Thus, we have identified two novel G13/LARG/rho-pathway inhibitor compounds, which have nM-μM potency. In addition to blocking transcription induced by rhoA, CCG-1423 also inhibits rhoC-stimulated luciferase expression (FIG. 6).

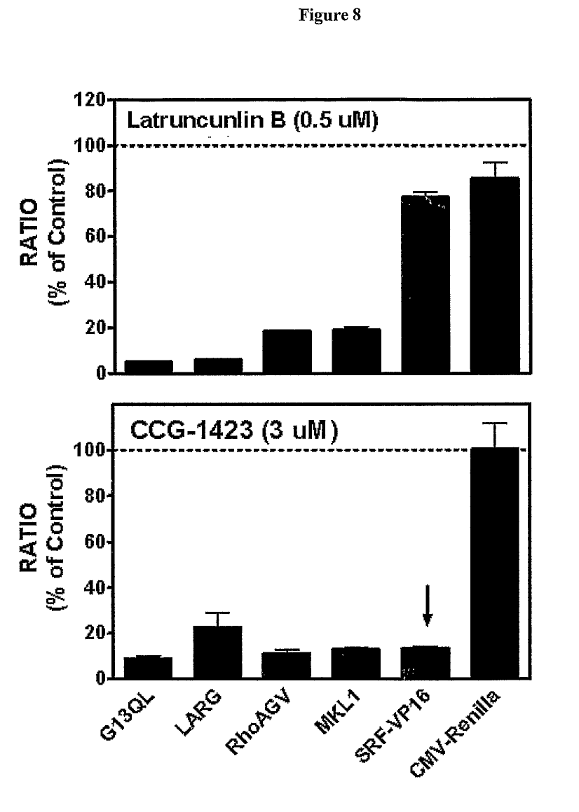

The point of action of these inhibitors in the assay was investigated by transfecting the cells with different activators of the pathway (rho, MKL1, and an activated SRF analog SRF-VP16). While the actin inhibitor Latrunculin B (which works downstream of rho) fully inhibits the luciferase expression induced by G13, LARG or constitutively active rhoA-GV, it did not inhibit expression activated by SRF-VP 16 (FIG. 8). This is consistent with the known site of action of Latruculin. In contrast, the novel inhibitors of the present invention, (as exemplified by CCG-1423) inhibited expression induced by G13, LARG, rhoA-GV, MKL, and SRF-VP16 indicating that these inhibitors have a different mechanism than does Latrunculin B. This was not just a non-specific effect since CCG-1423 at 3 μM did not inhibit CMV-Renilla expression and had only a slight inhibitory effect on a Gal-4-VP16 Luciferase signal. While the data in FIG. 8 suggest that SRF is being inhibited by the compounds of the present invention, subsequent studies have shown that the compounds inhibit MKL. Thus, the compounds may be acting by inhibiting MKL- or SRF- or both MKL and SRF-dependent gene transcription. The initial data suggest that the inhibition is greater for MKL-dependent gene transcription than for SRF-dependent gene transcription. Regardless of the mechanism of action, the present invention describes a novel gene transcription inhibitor that selectively prevents MKL/SRF-mediated signals.

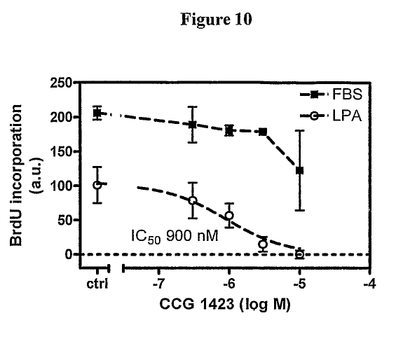

Even more exciting are the effects of the novel inhibitors of the present invention on PC-3 cell functions (LPA-stimulated DNA synthesis, proliferation, and invasion). More particularly, it is shown that for the highly aggressive human PC-3 prostate cancer cell line, the novel inhibitors described herein inhibit proliferation, LPA-stimulated DNA synthesis, and matrix invasion. All of these effects occur at remarkably similar concentrations (IC₅₀ 900, 830, and 600 nM, respectively). This is also very similar to the concentration needed to inhibit rho-stimulated SRE.L luciferase expression in HEK293T cells (980 nM). The compound is not just killing the cells because CMV-Renilla expression is not inhibited and DNA synthesis in the presence of serum is not inhibited until high concentrations are reached. Also, as a control for the BrdU incorporation studies in FIG. 10, WST1 metabolism was measured after 18 hours of drug treatment in the absence of serum and there was no decrease in viable cells even at 10 uM CCG-1423. Thus, CCG-1423 inhibits a wide variety of cellular functions in PC-3 prostate cancer cells but, interestingly, the SKOV-3 ovarian cancer cell line is significantly less sensitive. Only at high concentrations (10 uM) is there any effect on proliferation or matrix invasion and there is no inhibition of BrdU incorporation as well.

The compounds identified herein act at a distal site in gene transcription but have dramatic and specific effects on important cellular functions of the PC-3 prostate cancer cell. The potency (600-1000 nM) of these compounds is remarkable. As such, not only does the present invention provide details of methods of screening for effectors of rho-mediated pathways, the invention further provides examples of compounds that will be useful in medicaments and methods of effecting inhibition of a variety of cellular processes.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

1. A composition comprising an isolated compound that has a general structural formula (I): 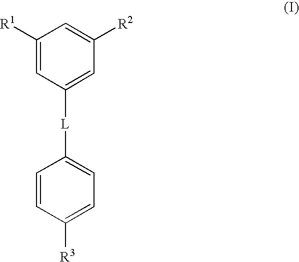
wherein R¹ and R², independently are selected from the group consisting of hydrogen, halo, C₁₋₃alkyl, C₁₋₃alkoxy, CF₃, OCF₃, C₁₋₂alkylOCH₂, NO₂, and CN, with the proviso that at least one of R¹ and R² is different from hydrogen;
R³ is selected from the group consisting of halo, C₁₋₃alkyl, C₁₋₃alkoxy, CF₃, OCF₃, C₁₋₂alkylOCH₂, NO₂, and CN: and
L is a linking group about four to about eight atoms or functional groups in length, wherein said compound is present in an amount effective to inhibit rho-mediated gene transcription.

2. The composition of claim 1, wherein said isolated compound of general formula I comprises two terminal hydrophobic phenyl groups, and a hydrophilic linking group L.

3. The composition of claim 1, wherein L is selected from the group consisting of C(═O), C(═S), NR^{a}, C═N(R^{a}), SO₂, SO, O, S, a phenyl ring and C(R^{b})₂ group,
wherein R^{a} independently, is hydrogen, hydroxy, or methyl and wherein R^{b}, independently, is hydrogen or methyl.

4. The composition of claim 1, wherein said isolated compound has the formula A or formula B:

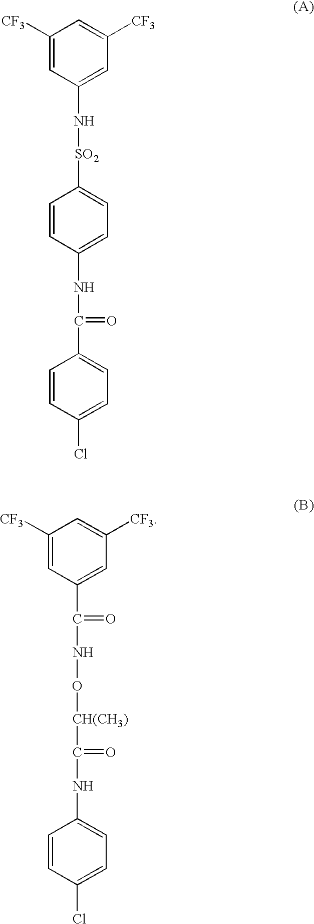

5. A pharmaceutical composition that comprises an isolated compound of claim 1 and a pharmaceutically acceptable carrier, excipient or diluent.

6. A method of inhibiting MKL-dependent gene transcription, SRF-dependent gene transcription, rho-stimulated gene transcription, LPA-stimulated DNA synthesis, or spontaneous matrix invasion of a cancer cell comprising contacting said cancer cell with an isolated compound of claim 1.

7. A method of inhibiting cancer cell growth comprising contacting a cancer cell with an isolated compound of claim 1.

8. The method of claim 7, wherein said cancer cell is located in vitro and said inhibition of cancer cell growth is in an in vitro assay.

9. The method of claim 7, wherein said cancer cell is located in vivo in an animal.

10. The method of claim 9, wherein said cancer cell is in a tumor and said inhibition of cancer cell growth comprises a decrease in tumor size.

11. The method of claim 9, wherein said cancer cell is a cancer cell that is potentially metastatic cancer cell and said inhibition of cancer cell growth comprises inhibiting or preventing metastatis of said cancer cell.

12. The method of claim 10, wherein said tumor is resected and said composition is contacted with said tumor prior to resection or during resection or said composition is contacted with said animal at the cavity of said tumor resection.

13. The method of claim 11, wherein said tumor is resected and said composition is contacted with said tumor prior to resection or during resection or said composition is contacted with said animal at the cavity of said tumor resection.

14. A method of inhibiting an inflammatory response in an animal comprising contacting said animal with an isolated compound of claim 1.

15. (canceled)

16. (canceled)

17. (canceled)


SEQUENCE LISTING (5 sequences)

SEQ ID NO: 1 - DNA, 4836 bp, Homo sapiens
ccatggagcagaagctgatctccgaggaggacctgggatccagtggcacacagtctactatcaccgacaggtttcccctcaaaaaacctataaggcatggaagtattttgaaccgagagtcaccaacagataagaagcagaaagttgagcgcattgcatcacatgattttgaccccacagatagctcctccaagaagacaaagtctagttcagaggagagtagatccgagatatatggtcttgttcagcgttgcgtaatcatccagaaagatgacaatggatttgggctgacggtcagtggagacagtccagtcttcgtacagtctgtcaaagaagatggagcagccatgcgggctggagtacagacaggtgatcgaatcatcaaggtgaatggaactctggtgactcattcaaatcatctggaggtggtgaagctaatcaaatctggttcctatgtagctctcactgttcagggacgcccacctgggtcgccccagattccacttgccgactctgaagtagagccgtcagtcattggacatatgtctcccatcatgacatctcctcattcacctggagcatctgggaatatggagagaatcactagtcctgtgctcatgggggaggaaaacaatgtggttcataaccagaaagtagaaattctgagaaaaatgttacagaaagaacaggaacggctacagttattgcaggaagattacaaccgaacacctgcccaaagattgctaaaagagatccaagaggccaagaaacacattcctcagctgcaagagcagttatccaaagccacaggctctgctcaggatggagctgtagttacaccctccagacctttaggggacaccctaacagtcagtgaggcagaaacagatcctggagatgtactgggcaggactgactgtagcagtggagatgcttctcggcccagtagtgacaatgcagatagtcccaagagtggcccaaaagagagaatttatctagaggaaaacccagagaaaagtgaaacaattcaggacactgacactcaatcacttgtcggaagtccctcaacccgtatagcacctcatattattggagcagaagatgatgattttggtactgaacatgaacagatcaatggacagtgcagctgtttccagagcattgaattactaaaatctcgcccggctcatttggctgttttcttacaccatgtagtttcacaatttgaccctgcgactttgctctgttatctctattcagacctgtataaacataccaattccaaagaaactcgtcgcatcttccttgagtttcatcagttctttctagatcgatcagcacacctgaaagtttctgttcctgatgaaatgtctgcagatctagaaaagagaagacctgagctcattcctgaggatctgcatcgccactatatccaaactatgcaagaaagagtccatccagaagttcaaaggcacttagaagattttcggcagaaacgtagtatgggactgaccttggctgaaagcgagctgactaaacttgatgcagagcgagacaaggaccgattgactttggagaaggagcggacatgtgcagaacagattgttgccaaaattgaagaagtattgatgactgctcaggctgtagaggaagataagagctccaccatgcagtatgttattctcatgtatatgaagcatttgggagtaaaagtgaaagagcctcgaaatttggagcacaaacggggtcggattggatttcttcccaaaatcaagcaaagtatgaagaaagataaagaaggggaagaaaaagggaagcgaagaggattccccagcatcctgggacccccacggagaccaagccgtcatgacaacagtgcaattggcagagccatggaactacagaaggtgcgccaccctaagcacttatccacaccctcatctgtgagtcctgaacctcaggactctgccaagttgcgccagagtgggttagcaaatgaaggaacagacgcaggatacctgcctgccaattccatgtcttctgtagcttcaggggcctctttttcccaggaaggagggaaagagaatgatacaggatcaaagcaagttggagaaacatcagcacctggagacaccttagatggcacacctcgtactctcaatactgtctttgatttcccaccacctccattagaccaagtgcaggaagaggaatgtgaagtagaaagggtgactgaacatgggacaccaaagccctttcgaaagtttgacagtgtagcttttggagaaagtcaaagtgaggatgaacaatttgaaaatgacttagagacagatccacccaactggcagcagcttgttagtcgagaagtgttactgggactaaaaccttgtgaaatcaaaagacaggaagtgattaatgaattgttctacactgaaagagctcatgttcgaacactgaaggttcttgatcaagtgttctatcagcgagtatccagagaaggaattctgtcaccctcagagctacggaaaattttttcaaacttggaagatattcttcaacttcatattggattgaatgaacaaatgaaggctgttcgaaagagaaatgagacctctgttatcgatcagattggggaagatttgctgacatggttcagcggaccaggagaggagaaattgaaacatgctgctgctaccttttgcagtaaccaacctttcgccctggaaatgatcaaatctcgtcagaaaaaggattctcgatttcagacttttgtgcaagatgctgaaagtaatccactgtgtcgtcgtcttcaactgaaggatattattcccactcaaatgcaaaggcttactaagtacccacttctgttggataatattgccaaatacacagaatggccaacagaaagggagaaggtgaagaaagctgcagatcactgtcgtcagatcttaaattttgtaaatcaggctgtcaaggaggcagaaaacaagcagcgcctagaagattatcagcgtcgccttgatacctccagcctgaagttgtcagagtacccaaatgttgaagagctcaggaatttggatttaacaaaaaggaagatgattcatgaagggccattggtttggaaggtgaatagagataaaactattgatttatacacgttgctgctggaagacattcttgtattgttacaaaagcaggatgatagactggttttaaggtgtcatagtaagattctggcatctacagctgatagcaaacacacgtttagccctgtcattaagttgagtacagtgttggttcgacaagtggcaacagataacaaagctttattcgtcatttccatgtcagacaatggcgctcagatttatgaactggtggcacagacagtttctgaaaagactgtctggcaggacctaatctgtcggatggctgcatcagtgaaggagcaatccacaaagccaattccattaccacagtcaacacctggcgaaggagataatgatgaagaagatccttcaaaattaaaagaggagcagcatggcatttcagtcactggtttgcagagtccagacagagatttgggattagaatctaccttaatatcgtcaaaacctcagtctcattcactgagtacctctgggaaatcagaggtacgtgatctgtttgtggctgagagacagtttgcaaaggaacaacatacagatgggacactaaaggaagttggagaagattatcaaatcgcaatcccagattcacacctgcctgtctcagaagaacggtgggcattggatgcactaagaaatttgggtttgttgaagcagttgctggtgcaacagctaggtttgactgagaagagcgttcaggaagactggcaacatttcccaagatacagaacagcctctcaggggccgcagacagacagtgtcatccagaactctgaaaatattaaggcctatcattctggtgaaggacatatgccctttagaactggaactggtgacattgcaacttgttacagtccacggacttcaactgaatcttttgctccacgggattcagtgggactggcaccccaggatagccaggcaagtaacattttagtaatggaccacatgattatgaccccagagatgcctaccatggagccagaagggggtcttgatgacagtggagagcacttttttgatgcccgtgaagcacatagtgatgagaatccatcagaaggtgatggagcagttaacaaggaagagaaggatgttaatttacgcatctcaggaaactatttgatccttgatggctatgacccagtgcaggagagttccacagatgaggaggttgcttcctcacttaccctgcagcccatgacaggcatccctgctgtggaatccacccaccagcagcaacattctcctcagaatactcactccgatggggcaatttcaccattcacccccgaatttctggtccagcagcgctggggagctatggagtattcctgttttgagatccagagtccctcctcttgtgcagattcacagagccagatcatggagtacattcataagatagaggctgaccttgaacacttaaagaaggtggaggaaagttacaccattctttgccaaaggctggctggatcagccctcacagacaagcactcagataaaagttagagccgcatgtcctggaggtgactgcaggttgttggatttggagtatcggccgtgtctcaccacatcctggctccagtgtggatgcagagagagtgtgacagaggatctgcctgtgaaccacctgggattagtcaagtcccaaggtgcccagagtgggactagt

SEQ ID NO: 2 - DNA, 1195 bp, Homo sapiens
gctagcgtttaaacttaagcttggtaccaccatggcggacttcctgccgtcgcggtccgtgctgtccgtgtgcttccccggctgcctgctgacgagtggcgaggccgagcagcaacgcaagtccaaggagatcgacaaatgcctgtctcgggaaaagacctatgtgaagcggctggtgaagatcctgctgctgggcgcgggcgagagcggcaagtccaccttcctgaagcagatgcggatcatccacgggcaggacttcgaccagcgcgcgcgcgaggagttccgccccaccatctacagcaacgtgatcaaaggtatgagggtgctggttgatgctcgagagaagcttcatattccctggggagacaactcaaaccaacaacatggagataagatgatgtcgtttgatacccgggcccccatggcagcccaaggaatggtggaaacaagggttttcttacaatatcttcctgctataagagcattatgggcagacagcggcatacagaatgcctatgaccggcgtcgagaatttcaactgggtgaatctgtaaaatatttcctggataacttggataaacttggagaaccagattatattccatcacaacaagatattctgcttgccagaagacccaccaaaggcatccatgaatacgactttgaaataaaaaatgttcctttcaaaatggttgatgtaggtggtctgagatcagaaaggaaacgttggtttgaatgtttcgacagtgtgacatcaatacttttccttgtttcctcaagtgaatttgaccaggtgcttatggaagatcgactgaccaatcgccttacagagtctctgaacatttttgaaacaatcgtcaataaccgggttttcagcaatgtctccataattctgttcttaaacaagacagacttgcttgaggagaaggtgcaaattgtgagcatcaaagactatttcctagaatttgaaggggatccccactgcttaagagacgtccaaaaattcctggtggaatgtttccggaacaaacgccgggaccagcaacagaagcccttataccaccacttcaccactgctatcaacacggagaacatccgccttgttttccgtgacgtgaaggatactattctgcatgacaacctcaagcagcttatgctacagtgatctagagatctctagagggcccgtttaaac

SEQ ID NO: 3 - DNA, 19 bp, Artificial sequence
  Synthetic primer
cataccatctctaccgacg

SEQ ID NO: 4 - DNA, 19 bp, Artificial sequence
  Synthetic primer
actgaagtctcggccagct

SEQ ID NO: 5 - DNA, 19 bp, Artificial sequence
  Synthetic primer
gaaactcgtcgcatcttcc